(19) United States
(12) Patent Application Publication
(10) Pub. No.: US 2017/0253921 A1
(43) Pub. Date: Sep. 7, 2017

(54) METHODS, KITS & COMPOSITIONS FOR DETERMINING GENE COPY NUMBERS

(71) Applicants: Life Technologies Corporation, Carlsbab, CA (US); Life Technologies Corporation

(72) Inventors: Chunmei LIU, Palo Alto, CA (US); Shoulian DONG, Mountain View, CA (US); Junko STEVENS, Menlo Park, CA (US)

(21) Appl. No.: 15/518,016

(22) PCT Filed: Oct. 13, 2015
(86) PCT No.: PCT/US2015/055344

§ 371 Date: Apr. 10, 2017

Related U.S. Application Data
(60) Provisional application No. 62/063,315, filed on Oct. 13, 2014.

(51) Int. Cl. C12Q 1/68

(52) U.S. Cl. CPC C12Q 1/6851; C12Q 1/686; C12Q 1/6853; C12Q 1/6883; C12Q 2537/16; C12Q 2537/161; C12Q 2545/107; C12Q 2600/16; C12Q 2545/113

(57) ABSTRACT

The present specification relates to methods, compositions, and kits for determining the presence of the number of copies of a gene. The present disclosure provides methods and compositions to determine zygosity in transgenic plants, number of transgenes in a plant or animal, karyotyping, determine CNV in human and animal samples, diagnose and detect diseases and conditions that are characterized by having variations in the number of copies of one or more genes.

### CROSS REFERENCE TO RELATED APPLICATIONS

This application claims a priority benefit under 35 U.S.C. §119(e) from U.S. Provisional Application No. 62/063,315 filed Oct. 13, 2014, which is incorporated herein by reference.

### FIELD

The present specification relates to compositions, methods and kits for determining the number of copies of one or more genes (e.g., such as but not limited to, number of transgene copies, zygosity, copy number variation (CNV) of one or more genes, and/or karyotyping).

### BACKGROUND

Determination of the number of copies of a gene present in a genome has several applications. Multiple copies of a transgene in a transfected cell causes varied phenotypic and physiological changes in the transfected cell. In other cases, an inherent or an acquired variation in the copy number of a gene, such as an increase or a decrease in the copy number of a gene, can be the cause of a genetic disease or an inherited defect.

In agricultural biology, determination of the copy number of a transgene that is introduced into a plant is required to assess the transgenic plant for desired characteristics. This is very important for both basic plant biology and industrial crop improvement. A plant expressing none, too few, or too many copies of a transgene will generally not have a desired characteristic such as phenotype, yield, insect/pest resistance, herbicide resistance as well as nutritional improvement. Following transformation with transgene(s), traditionally, Southern blot or T-DNA flanking sequence analysis is conducted. However, Southern blot analysis requires large amount of material which is not available at the early seeding stage and the T-DNA flanking sequence analysis is technically unstable. Current agricultural biology applications use methods that utilize real-time quantitative polymerase chain reaction (qPCR) methods to determine the number of copies of transgene(s). However, qPCR methods to determine zygosity (number of copies of a gene) require the use of real-time PCR instruments making the method expensive. Furthermore, real time qPCR methods are not well suited for high throughput screening to screen thousands and millions of transfected plant cells within a very short time period during breeding. In addition, the existing qPCR methods are not as sensitive for resolving gene copy numbers.

Human and animal cells have varied gene copy numbers of certain genes. In some cases, gene copy number variations (CNV) are the underlying cause of a disease/defect in the animal or human. Current methods used for CNV detections include comparative genomic hybridization (array CGH), fluorescence in situ hybridization (FISH), multiplex amplification probe hybridization, microarray as well as next generation sequencing. All of these methods are costly in terms of reagents, labor and time, and also require a considerable amount of DNA.

While real-time qPCR methods can be used to determine quantity gene copies, for heterogeneous specimens, more sensitive, better, cheaper and faster methods are desired in the art to determine the copy number of genes.

### SUMMARY

The present specification relates, in some embodiments, to methods, compositions and kits for determining the number of copies of a gene. In some embodiments, the present specification describes methods, compositions and kits for determining the copy number variation (CNV) of a gene comprising determining the number of copies of the gene with varying copy numbers.

In some embodiments, a method of the disclosure is a method for determining the number of copies of a gene and comprises: 1. contacting a sample for or from which the number of copies of a target gene are to be determined with: at least a first primer pair having the ability to selectively hybridize to portions of the target gene for which the number of copies are to be determined; at least a second primer pair having the ability to selectively hybridize to portions of a background nucleic acid sequence; and optionally, a third primer pair having the ability to selectively hybridize to portions of a reference nucleic acid sequence; 2. providing conditions for a nucleic acid amplification reaction to generate amplicons of: a) the target gene or fragment thereof, b) the background nucleic acid or fragment thereof, and optionally, c) the reference nucleic acid or fragment thereof; 3. analyzing the amplified amplicons; and 4. determining the copy number of the target gene. In some embodiments, the nucleic acid amplification reaction comprises a polymerase chain reaction (PCR).

In some embodiments, the number of copies of a gene can be zero, one, two, three, four, five, 6, 7, 8, 9, 10, 15, 20, 25, 30, 35, 40, 45, 50, 55, 60, 65, 70, 75, 80, 85, 90, 95, 100, 150, 200 or more and any number or range there between.

In some embodiments of a method of the disclosure, determining the copy number comprises determining the Rn/ΔRn values. In some embodiments, determining the copy number comprises end-point PCR.

In some embodiments of a method of the disclosure, determining the copy number comprises determining the Ct values. In some embodiments, determining the copy number comprises real-time qPCR.

In some embodiments, analyzing the amplicons comprises determining the Rn/ΔRn values. In some embodiments analyzing the amplicons comprises determining the Ct values.

In some embodiments a method of the disclosure for determining the number of genes, further comprises using one or more probes. In some embodiments, the one or more probes are labeled. Various types of labels that can be used with probes and their uses are described in sections below.

In some embodiments, the one or more probes are dually labeled. In one example embodiment a dually labeled probe can be labeled with a fluor and a quencher. In some embodiments, each probe used can be labeled with a different fluor and a different quencher, or can be labeled with the same quencher and a different fluor.

In some embodiments of the method of determining copy number of a gene, the one or more probes are used for detecting amplification, and/or for quantifying amplification and/or for both detecting and quantifying amplification. The same or different probes can be used for each of these.

In some embodiments of the method of determining gene copy number, probes and primers that are used can be all contacted simultaneously with the sample for which gene copy number is to be determined. In some embodiments of the present methods, the PCR is a multiplex PCR.

In some embodiments of a method of the disclosure, the PCR assay is a 5′nuclease assay. In a 5′ nuclease PCR assay a labeled probe is used.

In some embodiments of the method of determining gene copy number, the one or more probes are contacted with the PCR reaction after amplification is complete to selectively hybridize to amplicons and to detect various amplicons generated in the PCR.

In some embodiments of the method of determining gene copy number, one or more probes are contacted with the PCR reaction prior to amplification to selectively hybridize to one or more genes including: a target gene, a background gene and/or a reference gene, and to detect amplification; and one or more probes are contacted with the PCR reaction after amplification is complete to selectively hybridize to amplicons and to detect various amplicons generated in the PCR.

A background sequence can be any nucleic acid sequence that has no homology with the target gene of interest whose copy number is to be determined. In some embodiments, a background sequence can be any nucleic acid sequence that has no homology with any gene in the genome of the organism for which gene copy number of a target gene is to be determined. In some embodiments, a background sequence is provided externally. In some embodiments, a background sequence is an exogenous sequence. In some embodiments, a background sequence is internal or endogenous.

A wide variety of samples can be tested by methods of the disclosure. Non-limiting example samples that can be used to determine gene copy number include a nucleic acid, an isolated nucleic acid, a gDNA, DNA, RNA, mRNA, a chromosome, a cell, a cell lysate, a plant derived sample, including samples from plant leaves, stems, stalks, seeds, germ, plant tissue derived crude lysates, bacterial samples, fungal samples, viral samples, animal samples, human samples, samples derived from human/animal cells, tissues, bodily fluids such as blood, plasma, serum, whole blood, lymph, sweat, semen, bone marrow, urine, saliva , buccal swab, fecal matter, milk, tumors, cancers, circulating tumor cells, diseased tissues, samples obtained by biopsy, veterinary samples, skin samples, hair samples, crude lysates of any of the above, whole cells, and isolated nucleic acids of any of the above.

In one embodiment of the method of the disclosure, the sample is a crude cell lysate, a lysate, or a sample having varying amount of nucleic acids, and the sample is contacted with the third pair primers having the ability to selectively hybridize to nucleic acid sequences in a reference nucleic acid. Use of a reference nucleic acid serves to normalize the sample.

In some embodiments, the present disclosure describes a method called controlled plateau of PCR (referred to as CoP'ed PCR) which provides control of the plateau of PCR by running an additional PCR reaction in the background (also referred to herein as “background PCR” or “invisible PCR”), in addition to the PCR reaction to detect the target gene of interest for which copy number is to be determined (also referred to herein as the “target PCR”) and analyzing the data and comprises: 1) contacting a sample with: a) at least a first pair of PCR primers that have the ability to selectively hybridize to nucleic acid sequences in the target gene whose copy number is to be determined; b) at least a second pair of PCR primers that have the ability to selectively hybridize to nucleic acid sequences comprised in a background nucleic acid sequence (also referred to as a “background sequence” or “invisible sequence” in this specification); and c) optionally, at least a third pair of PCR primers that have the ability to selectively hybridize to nucleic acid sequences in a reference or control gene which acts as a positive control for the PCR reaction; 2) performing a polymerase chain reaction (PCR) to amplify the following: a) the target gene or a fragment thereof, b) the background sequence or a fragment thereof, and c) optionally the control gene or a fragment thereof; and 3) analyzing the products of amplification to determine the number of copies of the target gene. In some embodiments, the PCR is an end-point PCR. In some embodiments, the PCR is a real-time qPCR.

In some embodiments a method of the disclosure that comprises co-amplifying a background sequence, provides one or more of the following advantages when compared to a PCR method that does not co-amplify a background sequence, including: increasing the ability to resolve the presence of one or multiple copies of a gene, increasing the sensitivity of detection of multiple copy numbers of a gene, superior ability to detect gene copy numbers using end-point PCR methods of analysis, superior ability to detect gene copy numbers using real-time qPCR methods, decreasing the costs of copy number determination using end-point PCR, increasing the speed of detection, reduced turnaround time, and/or increased throughput.

In some embodiments, a method for determining copy number using end-point PCR, does not require a real time instrument to monitor the signal. In one embodiments, an end-point PCR reaction according to the disclosure can be run (carried out) on a thermocycler and the progress of amplification readings can be obtained on any fluor-reader (including but not limited to a real time instrument), which can greatly increase throughput for screening. Not using a real-time instrument saves the cost of having to purchase such an instrument.

In some embodiments, the present specification describes a method to determine the copy number of transgenes (also called zygosity). In some embodiments, the present specification describes a method to determine the copy number variations (CNV) of certain genes. In some embodiments, the present specification describes a method to determine the copy number of genes which serves to detect and diagnose diseases or conditions associated with gene copy number variation of the gene.

Some embodiments of the present disclosure describe compositions for a reaction mix comprising: at least a pair of target gene specific primers; a background nucleic acid sequence; at least a pair of primers specific to the background sequence; optionally, a pair of primers specific to a reference nucleic acid sequence; a DNA polymerase; dNTP's; MgCl₂; and one or more buffers.

A composition/reaction mix of the disclosure can further comprise one or more probes, wherein the probe(s) comprises a nucleic acid sequence operable to selectively hybridize to one or more of: a target nucleic acid sequence, a reference nucleic acid sequence, a background sequence, an amplicon or a fragment of an amplicon, wherein the amplicons can be a target gene amplicon or a fragment thereof, a reference amplicon or fragment thereof, a background sequence amplicon or a fragment thereof. In some embodiments, the composition of the disclosure can further comprise agents such as a Taq polymerase, VIP, antibody, dNTPs, glycerol, gelatin, albumin, ROX dye, NAN₃, Brij35, Tween 20, an emulsifier, a salt and one or more combinations thereof.

A composition/reaction mix of the disclosure can be used to perform a method to determine gene copy number of a gene. A composition/reaction mix of the disclosure can be used to perform a CoP'ed PCR method.

The present disclosure, in some embodiments describes kits comprising: at least a pair of target gene specific primers; a background nucleic acid sequence; at least a pair of primers specific to the background sequence; optionally, a pair of primers specific to a reference nucleic acid sequence, a DNA polymerase; dNTP's; MgCl₂; one or more buffers; and optionally, one or more probes, wherein one or more of the components are comprised in one or more containers and having instructions for using the kit. One or more compositions of a kit can be lyophilized. In some embodiments, all compositions of a kit of the disclosure will be lyophilized. In some embodiments, a kit of the disclosure with one or more lyophilized agents will be supplied with a re-constitution buffer. A kit of the disclosure can be used to perform a method to determine gene copy number of a gene. A kit of the disclosure can be used to perform a CoP'ed PCR method.

Some embodiments of the present disclosure may provide one or more technical advantages. One or more advantages of the methods, kits and compositions described above are increasing the ability to resolve one or multiple copies of a gene, increasing the sensitivity of detection of multiple copy numbers of a gene, superior ability to detect gene copy numbers using end-point PCR methods of analysis, superior ability to detect gene copy numbers using real-time qPCR methods, decreasing the costs of copy number determination, increasing the speed of detection, reduced turnaround time, and/or increased throughput as compared to other methods of the art currently used.

While specific advantages have been disclosed hereinabove, it will be understood that various embodiments may include all, some, or none of the previously disclosed advantages. Other technical advantages may become readily apparent to those skilled in the art in light of the teachings of the present disclosure. These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIGS. 1A and 1B depict data from the art that favor using real-time qPCR methods that use Ct (referred to as Cq) based gene copy number determination (see FIG. 1A) rather than end-point PCR methods of the art that use Rn based gene copy number determination (see FIG. 1B) due to the problem of PCR saturation or plateau in the latter;

FIG. 2 depicts a schematic representation of one embodiment of the present method comprising running a PCR in the background (referred to in FIG. 2 as “invisible PCR brake” or in the specification as “background PCR”) which slows the target and reference PCR and controls the PCR saturation at the end, the method is referred to herein as controlled plateau of PCR (CoP'ed PCR), according to one embodiment of the disclosure;

FIGS. 3A and 3B depict exemplary CoP'ed PCR method results which reduced plateau of target PCR gDNA as compared to standard PCR methods (STD), separating 1, 2 and 3 copies of a target gene, wherein two different exemplary assays (one assay shown in FIG. 3A and the second assay shown in FIG. 3B) were tested with a fixed amount of purified gDNA, according to one embodiment of the disclosure;

FIGS. 4A and 4B depicts an example method of a CoP'ed PCR assay which reduced plateau of target PCR as compared to standard PCR methods (STD), wherein two different exemplary assays (one assay shown in FIG. 4A and another assay FIG. 4B) were tested each having different amounts of purified human gDNA as sample mixed with a corn crude lysate, according to one embodiment of the disclosure;

FIG. 5 depicts an example CoP'ed PCR method for better end-point gene copy number separation with corn crude samples after normalization of input amount, according to one embodiment of the disclosure; and

FIGS. 6A and 6B depict an example CoP'ed PCR method improving copy number separation from human crude blood sample for both real time Ct based copy number (copy#) detection and end-point Rn based copy# detection, performed on samples from two individuals (FIG. 6A and FIG. 6B), according to one embodiment of the disclosure.

It is to be understood that both the foregoing general description and the following detailed description are exemplary and explanatory only and are not intended to limit the scope of the current teachings. The section headings used herein are for organizational purposes only and are not to be construed as limiting the subject matter described in any way.

### DETAILED DESCRIPTION

Determining the number of copies of a gene is important for several applications. In agricultural applications, zygosity information is required for effective breeding and colony maintenance of a transgene of interest. For example, if a transgene of interest is not incorporated into the genome of a plant, the plant will not have the desired phenotype. On the other hand having too many copies of a transgene may also not produce a desired phenotype.

A transgenic plant may incorporate 0, 1, 2, 3, 4, 5 . . . . or more copies of the transgene of interest. The copy number of a transgene determines the phenotype and other properties of the plant. Hence, knowing the transgene copy number is critical to the characterization and selection of candidate transgenic plants for breeding and cultivation purposes.

Another area in which determining the copy number of a gene is important is in human and animal diseases or inherited conditions that are caused by gene copy number variations (CNV). The copy number of genes involved can be either increased or reduced (such as by a deletion). Exemplary non-limiting conditions include Down's syndrome which is caused by trisomy of the chromosome 21, several cancers caused by aneuploidy of various tumor related genes, for example, high copies of ERBB2 is associated with aggressive forms of breast cancer, therefore, measuring the ERBB2 copy number can provide a diagnostic tool for breast cancer and other cancers. Similarly, copy number variations were identified on chromosome 22 in regions involved with spinal muscle atrophy and DiGeorge syndrome, as well as in the imprinted chromosome 15 region associated with Prader-Willi syndrome and Angelman syndrome. These diseases might be caused by copy number variants due to inversions and deletions in critical genes. Copy number variants have also been detected in genetic regions associated with complex neurological diseases, such as Alzheimer's disease and schizophrenia.

Existing methods to determine CNV's typically include cytogenetic methods such as fluorescent in situ hybridization, comparative genomic hybridization, and/or virtual karyotyping with SNP arrays. Other methods include next-generation sequencing and quantitative PCR (qPCR), paralog-ratio testing (PRT) and molecular copy number counting (MCC). qPCR compares threshold cycles (Ct) between the target gene and a reference sequence with normal copy numbers, to generate ΔCt values which are used for CNV calculation. This method has been used in large-scale CNV analysis in detecting disease associations, for example, psoriasis and Crohn's disease. With the development of genome-wide CNV screening, qPCR is often used as a confirmation method for computationally identified loci. Other multiplex PCR-based approaches, such as multiplex amplifiable probe hybridization, multiplex ligation-dependent probe amplification, multiplex PCR-based real-time invader assay, quantitative multiplex PCR of short fluorescent fragments, and multiplex amplicon quantification, have also been used for targeted screening and validation of CNVs.

For zygosity determination in plants, current real-time quantitative PCR (qPCR) based copy number determination relies on measuring cycle of threshold (Ct/ΔCt), which requires real time instruments. Current qPCR methods lack the required sensitivity.

For example, real time qPCR methods rely on determining the Cq value (also referred to as Ct which is described later) upon which copy number determination is based. Determining the Ct or Cq value requires a real time PCR instrument which is expensive and not suitable for high throughput screening which requires the screening of thousands and millions of transfected plant seeds and leaves within a very short time period during breeding. In addition, existing Cq based qPCR methods require DNA purification which is tedious, costly and labor intensive.

In some embodiments, the CoP'ed PCR methods described herein can determine copy number of genes using crude lysates of samples (e.g., crude lysates from plants or crude lysates from animals), which simplifies the workflow and allows for a further cost-effective and time-saving method.

The present inventors have developed a PCR based method for determining copy number of genes for applications such as, but not limited to, zygosity and CNV determination, which provides one or more of the following: very good resolution of copy numbers, providing a high sensitivity, resulting in an increased throughput, reduced cost and improved turn-around time.

In some embodiments, the present methods comprise end-point PCR readings comprising measuring the value (Rn/ΔRn) where Rn is the measure of a reporter signal. As used herein, the terms “ΔRn” or “dRn” or “delta Rn” are interchangeable and refer to the difference in the normalized reporter signal (Rn) subtracted from the background signal (baseline) which is then normalized by a passive reference signal. ΔRn can be determined by the formula [Rn⁺−Rn⁻], where Rn⁺ is the Rn value for a PCR reaction involving all components, including a target nucleic acid to be amplified (also called as template), and Rn⁻ is the value for an unreacted sample, i.e., a PCR reaction involving all components except the target nucleic acids (no template).

A major challenge for determining copy number of a gene using end-point PCR is the saturation control of PCR. The saturation control of end-point PCR can be understood by analyzing what happens during a PCR reaction. A basic PCR run can be broken up into three phases: 1) the exponential phase characterized by an exact doubling of amplified product at every PCR cycle, assuming 100% reaction efficiency. The exponential phase reaction is specific and precise; 2) the linear phase wherein reaction components are being consumed, the reaction is slowing, and products are beginning to degrade. The linear phase has high variability; 3) the plateau phase where the reaction has stopped, no more products are being made and if left long enough, the amplified PCR products will begin to degrade. In end-point PCR the saturation phase is the phase used to analyze the amplified product (for example but not limited to by traditional gel detection, fluorescent plate readers and the like).

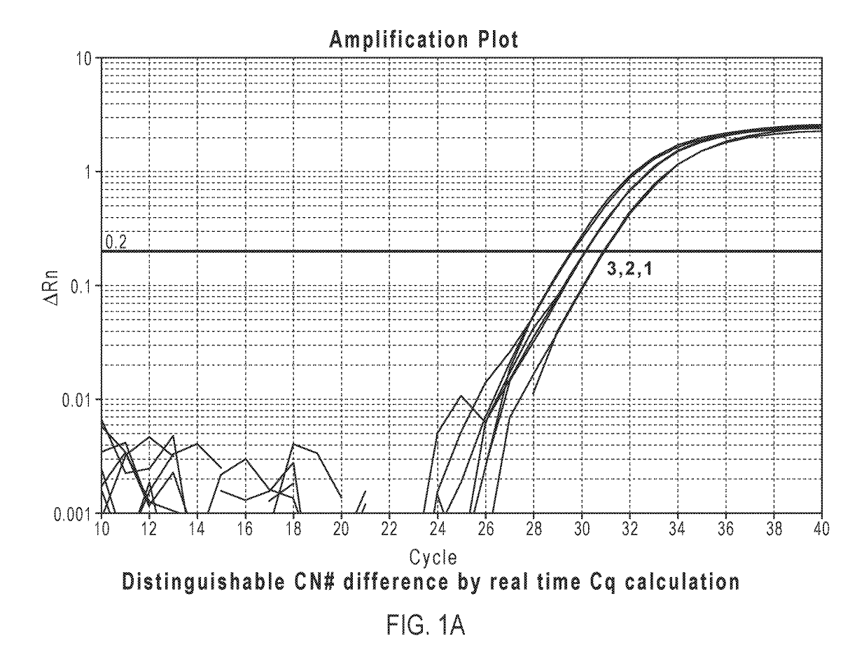

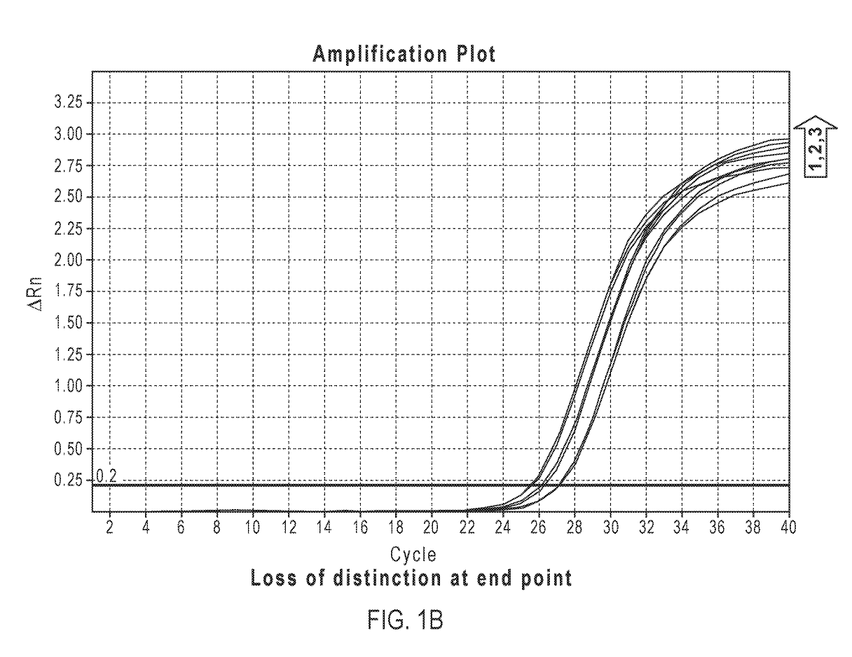

The differences between using real-time qPCR assays and end-point PCR assays that exist in the art for determining copy number of a gene are illustrated in an example shown in FIGS. 1A and 1B. FIG. 1A. depicts a real-time (Ct or Cq) based copy number (also referred to herein alternatively as CN#, copy#, or CN) determination with horizontal cutoff using the log scale. For pure, homogenous samples, Ct based copy# analysis is able to segregate 1, 2 and 3 copies of chromosome or genes. FIG. 1B. depicts end-point PCR (Rn) based copy# determination using end-point methods of the art. At the end of PCR, all curves (1, 2, 3 or more copies) are mixed together due to the saturation (plateau phase) and it is impossible to quantitate copy#.

Quantitation of a target nucleic acid by PCR is typically done by real-time qPCR methods. Theoretically, there is a quantitative relationship between amount of starting target sample and amount of PCR product at any given cycle number. Real-Time qPCR detects the accumulation of amplicon during the amplification reaction using real-time instruments. The data for real-time qPCR is measured at the exponential phase of the PCR reaction, which is the stable phase. See for example FIG. 1A.

However, end-point PCR methods use the plateau phase for gathering data, which for reasons described above is not the optimal phase for quantitation. See for example FIG. 1B. Detection in end-point is by the use of agarose gels, fluorescent plate readers, or other post PCR detection methods, which are not as expensive as using real-time instruments.

The present inventors then developed a method called controlled plateau of PCR (referred to as CoP'ed PCR) which provides control of the plateau of PCR by running an additional PCR reaction in the background (also referred to herein as “background PCR” or “invisible PCR”), in addition to the PCR reaction to detect the target gene of interest for which copy number is to be determined (also referred to herein as the “target PCR”) and analyzing the data.

In some embodiments, CoP'ed PCR analysis is by end-point data analysis. The present inventors have shown that CoP'ed PCR showed best separation of gene copy number through end-point PCR data analysis.

In some embodiments CoP'ed PCR data analysis is by real-time qPCR. In some embodiments, the present disclosure provides qPCR methods that provide more sensitive resolution of gene copy numbers. In some embodiments, the CoP'ed qPCR methods of the present disclosure provide more sensitive resolution of gene copy numbers as compared to the existing qPCR methods that do not comprise CoP'ed PCR. The present inventors have demonstrated that controlled plateau of PCR (referred to as CoP'ed PCR) results in better resolution of copy numbers in qPCR methods. Accordingly, in some embodiments a CoP'ed PCR method comprises running an additional PCR reaction in the background (also referred to herein as “background PCR” or “invisible PCR”), in addition to the PCR reaction to detect the target gene of interest for which copy number is to be determined (also referred to herein as the “target PCR”) and analyzing the data in real-time showed enhanced separation of gene copy number through qPCR data analysis.

In the CoP'ed methods of the disclosure, the additional PCR reaction (i.e., the background PCR) is to a nucleic acid sequence (also called a background sequence) that has no homology to the target gene of interest whose copy number is to be determined. This background PCR reaction competes for PCR reagents and enzymes with the target PCR, therefore, it slows down the target PCR. This controls the saturation of target and reference PCR and provides enhanced resolution of the target gene amplicons by providing greater resolution of target gene copy numbers.

In one embodiment, a CoP'ed method of the disclosure comprises: 1) contacting a sample that has a target gene whose copy number is to be determined with: a) at least a first pair of PCR primers that have the ability to selectively hybridize to nucleic acid sequences in the target gene whose copy number is to be determined; b) at least a second pair of PCR primers that have the ability to selectively hybridize to nucleic acid sequences comprised in a background nucleic acid sequence (also referred to as a “background sequence” or “invisible sequence” in this specification); and c) optionally, at least a third pair of PCR primers that have the ability to selectively hybridize to nucleic acid sequences in a reference or control gene which acts as a positive control for the PCR reaction; 2) performing a polymerase chain reaction (PCR) to amplify the following: a) the target gene or a fragment thereof, b) the background sequence or a fragment thereof, and c) optionally the control gene or a fragment thereof; and 3) analyzing the products of amplification to determine the number of copies of the target gene. In some embodiments, the PCR is an end-point PCR. In some embodiments, the PCR is a real-time qPCR.

In some embodiments, the method can further comprise using one or more probe to detect the products of amplification. In some embodiments, one or more probes is labeled. In some embodiments, one or more of the probes is a dual labeled probes. In some embodiments, the one or more of the probes used is labeled with a fluor and a quencher. In some embodiments, each probe used is dually labeled with a different fluor and a different quencher. In some embodiments, each probe used is dually labeled with a different fluor and the same quencher.

In some embodiments, the probes and the primers are all contacted simultaneously with the sample for which gene copy number is to be determined. In some embodiments, the probes and the primers are all contacted sequentially with the sample for which gene copy number is to be determined.

In some embodiments, one or more probes can be contacted with a sample prior to amplification along with the primers and other PCR reagents. In some embodiments, one or more probes can be contacted with the PCR reaction after amplification is complete to selectively hybridize to amplified amplicons and aid in detection of various amplicons generated in the PCR. In some embodiments, probes can be used for both the embodiments described above (i.e., one or more probes can be used for the amplification step (such as in a 5′nuclease assay) and one or more probes can be used for detecting the amplicons formed after amplification during the detection step). Probes used in each step can have the same or a different nucleic acid sequence.

In some embodiments the PCR assay comprises a 5′nuclease assay. In some embodiments, the PCR assay comprises a TaqMan® assay.

In one embodiment of a CoP'ed method as described above, the first pair of PCR primers are designed in reference to a target gene whose copy number is to be determined and comprise a forward and a reverse primer that can selectively hybridize to portions of the target gene and amplify the target gene or a fragment thereof when provided PCR amplification reaction conditions and components. The second pair of PCR primers are designed to a background sequence and comprise a forward and a reverse primer that can selectively hybridize to portions of the background nucleic acid sequence and amplify the background sequence or a fragment thereof when provided PCR amplification reaction conditions and components.

A background sequence in accordance to this disclosure can be any nucleic acid sequence that has no homology with the target gene of interest whose copy number is to be determined (including for example, transgenes, genes with CNV etc.).

In some embodiments, a background sequence is provided externally that has no homology with target gene. In some embodiments, a background sequence has no homology with any part of the genome of the organism tested. In some embodiments, a background sequence is an exogenous sequence.

In some embodiments, a background sequence can from about 5 kb to about 100 bp in length. In some embodiments, a background sequence can from about 5 kb, 4 kb, 3 kb, 2 kb, 1 kb, 500 bp, 400 bp, 300 bp, 200 bp, to about 100 bp in length including any lengths in between these numbers. In some embodiments, a background sequence can be from about 100 bp to about 150 bp, from about 100 bp to about 200 bp, and about 100 bp to about 300 bp, about 100 bp to about 200 bp, about 100 bp to about 1 kb, about 100 bp to about 2 kb, about 100 bp to about 3 kbp, about 100 bp to about 4 kb, or about 100 bp to about 5 kb in length. In some embodiments, the background sequence can be as short as 150 bp.

In one non-limiting exemplary embodiment, a background sequence is a KAZ plasmid sequence (Life Technologies, Cat# 4308323, TagMan® Exogenous Internal Positive Control Reagents) which has no homology with plant, human as well as other animal genomes. In this example embodiment, the amplicon size of the background PCR is between 100 to 150 bp.

In some embodiments, a background sequence amplicon size can from about 100 bp to about 300 bp in length. In some embodiments, a background sequence amplicon size can from about 100 bp to about 150 bp, from about 100 bp to about 200 bp, and about 100 bp, about 125 bp, about 150 bp, about 175 bp, about 200 bp, about 225 bp, about 250 bp, about 275 bp, or about 300 bp in length including any lengths in between these numbers. In some embodiments, the background sequence amplicon can be as short as 150 bp.

However background sequences and their amplicons may be of other varying lengths and one of skill in the art will recognize that the present embodiments are not limited to the lengths or specific sequences disclosed above which are provided as examples.

An optional third primer set maybe used to amplify a reference or a control nucleic acid sequence. In some embodiments a “reference” or “control” nucleic acid can comprise a passive or active signal used to normalize experimental results. In some embodiments, endogenous controls are examples of active references. Active reference means the signal is generated as the result of PCR amplification. The active reference has its own set of primers. An “endogenous control” refers to a DNA that has same copy in each cell, therefore, presumably, same copies with same amount of sample DNA input in each experiment. By using an endogenous control as an active reference, one can normalize the sample input and accurately determine the copy# after normalization. In some embodiments, whether or not an active reference is used, a passive reference (for example, containing the dye ROX) is used in order to normalize for non-PCR-related fluctuations in fluorescence signal.

Running the PCR amplification using the background sequence is able to slow down the saturation of the target gene PCR amplification. The present inventors have optimized background PCR (CoP'ed PCR) to achieve end-point PCR based determination of copy number of genes without impacting real time performance.

In some embodiments, when performing a CoP'ed PCR method, a certain ratio of background PCR nucleic acid template versus target PCR nucleic acid template is maintained. In some embodiments of a CoP'ed PCR method, a range of from about 100 to about 10,000 fold of background template nucleic acid as compared to target genome template nucleic acid is maintained. Accordingly, in some embodiments a range of “background template nucleic acid”:“target template nucleic acid” can be from about 100:1, 150:1, 200:1, 250:1, 300:1, 350:1, 400:1, 450:1, 500:1, 550:1, 600:1, 700:1, 800:1, 900:1, 1000:1, 1100:1, 2000:1, 3000:1, 4000:1, 5000:1, 6000:1, 7000:1, 8000:1, 9000:1 to about 10,000:1, including ratios there between. Ratios there between would include numbers such as 110:1, 2200:1, 5500:1 and the like.

In some embodiments, the length of an amplicon of a “background target” is similar to the length of an amplicon of a target nucleic acid. In some embodiments, the length of an amplicon of a “background target” is from about 10, 20, 30, 40 to about 50 nucleotides smaller or larger than the length of an amplicon of a target nucleic acid.

In some embodiments, the concentration range of PCR primers in a CoP'ed PCR reaction mixture is from about 0.5 μM to about 2 μM. In some embodiments, the concentration range of PCR primers in a CoP'ed PCR reaction mixture is from about 0.5 μM, about 1 μM, about 2 μM, about 3 μM, about 4 μM, about 5 μM, about 6 μM, about 7 μM, about 8 μM, about 9 μM to about 10 μM.

In some embodiments of a CoP'ed PCR, for a purified DNA sample, an input of purified sample DNA is from about 1 ng to about 100 ng. In some embodiments of a CoP'ed PCR, for a purified DNA sample, an input of purified sample DNA is from about 1 pg to about 1 ng, including ranges there between, for example 1 pg, 2 pg, 3 pg, 4 pg, 5 pg, 6 pg, 7 pg, 8 pg, 9 pg, 10, pg, 15 pg, 20 pg, 25 pg, 30 pg, 35 pg, 40 pg, 45 pg, 50 pg, 100 pg, 200 pg, 300 pg, 400 pg, 500 pg, 600 pg, 700 pg, 800 pg, 900 pg, to about 1 ng and ranges there between.

In one non-limiting embodiment of a CoP'ed PCR, while using a purified DNA template as a sample for the target gene, a triplex PCR mixture can comprise: a sample background DNA template input of from about 10 pg (or about 3 million copies) in combination with about 1 μM of PCR primers comprising background specific primers, about 1 μM of PCR primers comprising target specific primers and about 1 μM of PCR primers comprising control specific primers. In the above embodiments, the “background template” concentration is from about 100 to about 10,000 fold more than the target DNA template.

In some embodiments of a CoP'ed PCR, for a crude sample, the background template concentration and the primer concentrations can be from about half of the concentrations described above for purified DNA sample.

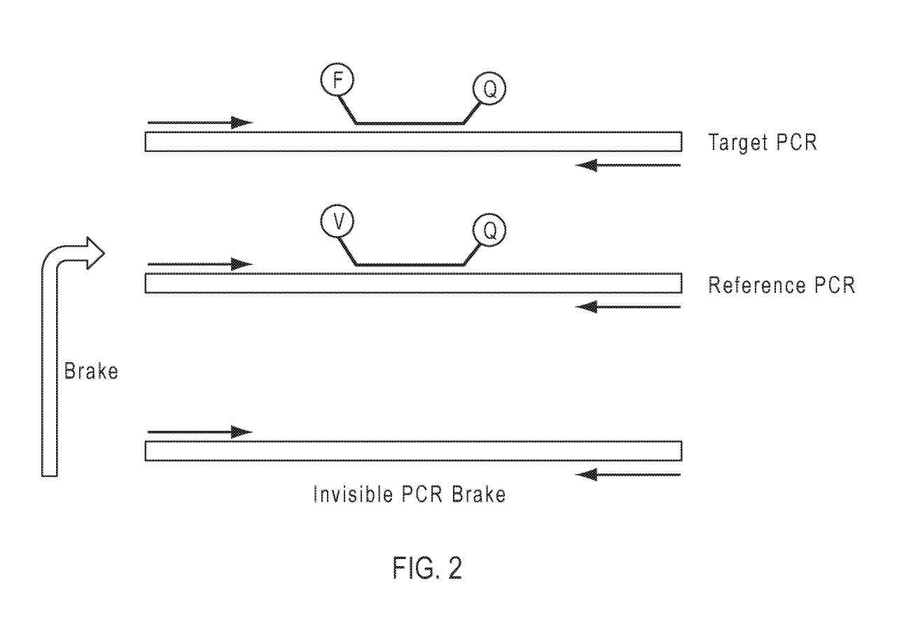

A non-limiting example schematic of an exemplary CoP'ed PCR of the present disclosure is shown in FIG. 2. As can be seen in the example of FIG. 2, a multiplex PCR reaction is carried out with a first “target PCR” amplification targeting the amplification of a “target sequence” which is the gene whose copy number is to be determined. A pair of primers, comprising a forward primer and a reverse primer, are depicted that selectively hybridized to regions comprised in the target sequence to prime the “target” amplification. In this example, a “target” detector probe dually labeled with a flour and quencher pair is also depicted (e.g., TaqMan® probe). In this example, a 5′ nuclease PCR assay is carried out to amplify the target sequence by a PCR reaction mix to produce amplicons comprising target sequences.

FIG. 2. also depicts a second “reference PCR” amplification targeting a reference or control nucleic acid sequence and a primer pair comprising a forward and a reverse primer targeting regions of a reference gene are depicted. In this example, a “reference” detector probe dually labeled with a flour and quencher pair is also depicted (e.g., TaqMan® probe). In this example, a 5′ nuclease PCR assay to amplify the reference sequence is carried out by a PCR reaction mix to produce amplicons comprising reference sequences. Simultaneous detection of both “target” amplicon and “reference” amplicon are possible by having two different fluors on the “target detector probe” and the “reference detector probe.”

FIG. 2. also depicts a third PCR reaction, the “background” PCR that is running in the background on a “background sequence.” This reaction, though not visualized in the present example, happens invisibly in the background with the first and second PCR reactions as described in the two paragraphs immediately above and allows control of plateau. The background sequence is an exogenous artificial template with no homology to the testing genome. The running of the background PCR in the background slows down the target PCR due to the competition of the substrates and enzyme, which then control the saturation of target PCR.

In one example embodiment (not depicted) a 3 kb background sequence was added into a testing sample together with PCR primers to amply a 120 bp sequence in the background.

FIG. 2 depicts a non-limiting example CoP'ed method for detecting a target nucleic acid and a reference nucleic acid using TaqMan® probes. However, a CoP'ed method can also be performed by hybridization with labeled probes upon the completion of PCR. A DNA double strand binding dye based detection method such as SYBRGreen® cannot be used since it captures the background PCR signal along with the target and control PCR signals.

One of skill in the art will recognize that the schematic example in FIG. 2 is merely provided as an example and is not to be construed as limiting the present disclosure. Other variations of the CoP'ed PCR method as described herein are possible.

In some embodiments, the present CoP'ed (end-point or real-time) PCR methods can be done on purified gDNA samples. In some embodiments, the present CoP'ed (end-point or real-time) PCR methods can be done on purified DNA samples. In other embodiments, the present CoP'ed (end-point or real-time) PCR methods can be done on intact cells and/or crude cell lysates which comprise target template DNA.

Other non-limiting exemplary DNA samples on which a method of the disclosure can be performed include, but are not limited to, genomic DNA, plasmid DNA, phage DNA, nucleolar DNA, mitochondrial DNA, chloroplast DNA, cDNA, synthetic DNA, chromosomal DNA, yeast artificial chromosomal DNA (“YAC”), bacterial artificial chromosome DNA (“BAC”), other extrachromosomal DNA, and primer extension products. Nucleic acid sequences also include, but are not limited to, analogs of both RNA and DNA.

In some embodiments, crude lysate from plant tissues were used to determine zygosity. For example, in a non-limiting example crude lysate of corn leaves were used. In some embodiments, human blood samples were used.

A wide variety of samples comprising a target gene whose copy number is to be determined can be used in methods of the disclosure and include without limitation any plant derived sample, including plant leaves, stems, seeds, germ, or plant tissue derived crude lysates, whole cells and/or isolated nucleic acids therefrom. Samples of the disclosure also include bacterial samples, fungal samples, viral samples, animal samples and human samples. Animal and human samples can be derived from any animal or human to be tested and may include cells, tissues, bodily fluids (such as but not limited to blood, plasma, serum, whole blood, lymph, sweat, semen, bone marrow, urine, fecal matter, milk), tissue/tumor/cancer/other disease samples obtained by biopsy, veterinary samples, skin samples, hair samples, crude lysates of any of the above, and/or isolated nucleic acids from any of the foregoing. Isolated nucleic acids include nucleic acids such as, but not limited to, genomic DNA (gDNA), DNA, RNA, mRNA, plasmid DNA, phage DNA, nucleolar DNA, mitochondrial DNA, chloroplast DNA, cDNA, synthetic DNA, chromosomal DNA, yeast artificial chromosomal DNA (“YAC”), bacterial artificial chromosome DNA (“BAC”), and other extrachromosomal DNA.

The present inventors were able to demonstrate that by running the background PCR (CoP'ed PCR), they were surprisingly able to achieve end-point segregation of copy numbers of genes. In addition, surprisingly unexpected improvement of real-time separation of copies of target genes in samples was seen.

Accordingly, one embodiment provides a method for screening for plants to determine zygosity. A method for screening plants to determine zygosity, according to one embodiment, comprises: 1) contacting a plant derived sample that has a target gene (e.g., a transgene) whose copy number is to be determined with: a) at least a first pair of PCR primers that have the ability to selectively hybridize to nucleic acid sequences in the target gene whose copy number is to be determined; b) at least a second pair of PCR primers that have the ability to selectively hybridize to nucleic acid sequences in a background sequence; and c) optionally, at least a third pair of PCR primers that have the ability to selectively hybridize to nucleic acid sequences in a reference or control gene which acts as a positive control for the PCR reaction; 2) performing an CoP'ed PCR reaction to amplify: the target gene or a fragment thereof, the background sequence or a fragment thereof, and optionally the control gene or a fragment thereof; and 3) analyzing the products of amplification to determine the number of copies of the target gene. The PCR reaction can be a duplex, triplex, or a multiplex reaction. The PCR reaction can be analyzed using end-point analysis or real-time qPCR analysis.

In some embodiments, a method of the disclosure is described as an end-point zygosity method. In some embodiments, crude samples can be used to streamline an end-point zygosity workflow.

Some embodiments describe duplexed assays (or multiplexed assays) to quantitate the level of a transgene relative to an endogenous normalization gene. A saturation control for a duplex assay (or multiplexed assay) is achieved by running a background PCR assay. In some embodiments, the PCR efficiency of the duplex (or multiplex) assays has to be similar and the saturation control from the background PCR assay has to be similar for each assay of the duplex or multiplex assays.

One embodiment of the disclosure describes diagnostic methods for detecting human gene copy number variation/aberration/increase/decrease using as a sample a human cell, a human tissue or a human derived nucleic acid sample. Human samples can include cells, tissues, bodily fluids (such as but not limited to blood, plasma, serum, whole blood, lymph, sweat, semen, bone marrow, urine, fecal matter, milk), tissue/tumor/cancer samples obtained by biopsy, skin samples, hair samples, crude lysates of any of the above, and/or isolated nucleic acids from any of the foregoing. In one embodiment, the human sample is a blood samples. In some embodiments the methods provide an increased sensitivity of detection of human gene copy number as compared to existing methods.

A diagnostic method for detecting human gene copy number aberration, according to one embodiment, can comprise: 1) contacting a human derived sample that has a target gene whose copy number is to be determined (e.g., a gene that is susceptible to CNV or a gene that is suspected to have CNV) with: a) at least a first pair of PCR primers that have the ability to selectively hybridize to nucleic acid sequences in the target gene whose copy number is to be determined; b) at least a second pair of PCR primers that have the ability to selectively hybridize to nucleic acid sequences in a background sequence; and c) optionally, at least a third pair of PCR primers that have the ability to selectively hybridize to nucleic acid sequences in a reference or control gene which acts as a positive control for the PCR reaction; 2) performing an PCR reaction to amplify the target gene or a fragment thereof, the background sequence or a fragment thereof and optionally the control gene or a fragment thereof; and 3) analyzing the products of amplification to determine the number of copies of the target gene. In some embodiments the analysis of data can be using end-point PCR data analysis or by using qPCR data analysis or both.

In some embodiments, the disclosure provides a method to diagnose trisomy of chromosome 21.

In some embodiments, the disclosure provides methods to diagnose a cancer comprising detecting aberrant numbers of copies of one or more genes that have an aberrant copy number in a cancer. One non-limiting example is the ERBB2 gene, high copies of which are associated with aggressive forms of breast cancer. Measuring the ERBB2 copy number using CoP'ed PCR methods of the present disclosure can provide a diagnostic tool for breast cancer.

One embodiment of the disclosure describes a diagnostic method for detecting animal gene copy number aberration using animal cell, tissue or nucleic acid samples. In one embodiment, the animal sample is a blood sample, a sample as described in sections above or any veterinary sample.

The methods of the present disclosure provide a cost-saving measure compared to current methods in the art that rely on real-time PCR methods and real-time instruments which are expensive. This is especially the case when there is a need to screen large numbers of samples for high throughput screening, such as in the case of transfected plants.

The present disclosure also describes compositions for a reaction mix for performing CoP'ed PCR methods to determine copy numbers of a gene. In some embodiments a composition/reaction mix for CoP'ed PCR comprises one or more of: at least a pair of target gene specific primers, a background sequence, at least a pair of primers specific to the background sequence, optionally, a pair of primers specific to a control or reference sequence, a DNA polymerase, dNTP's, MgCl₂ and one or more buffers. A composition/reaction mix can also comprise one or more probes, wherein the probe comprises a nucleic acid sequence operable to selectively hybridize to: a target nucleic acid sequence, a reference/control nucleic acid sequence and/or to an amplicon or a fragment of an amplicon, wherein the amplicon includes a target gene amplicon or a fragment thereof, a reference/control amplicon or fragment, and optionally to hybridize selectively to a background sequence and/or to an amplicon or a fragment of an amplicon of a background sequence.

A composition/reaction mix comprises probes of the disclosure which include probes to perform a 5′ nuclease assay and/or one or more probes to detect the products of amplification. In some embodiments, one or more probe of the composition/reaction mix is labeled. In some embodiments, one or more probes of the composition/reaction mix is a dual labeled probe. In some embodiments, one or more of the probes of the composition/reaction mix is labeled with a fluor and a quencher. In some embodiments, each probe of a composition/reaction mix is dually labeled with a different fluor and a different quencher. In some embodiments, each probe of a composition/reaction mix is dually labeled with a different fluor and the same quencher.

A composition/reaction mix for a CoP'ed PCR method can also comprise one or more agents such as: glycerol from about 3% to about 25% which can improve the PCR performance by stabilizing the enzyme as well as reducing Tm of difficult sequences; gelatin from about 0.01% up to about 2%, such as for example, bovine gelatin, fish gelatin; Tween 20 from about 0.001% to about 0.1%; Tris-HCL from about 0.01M to about 0.1M; MgCl₂ from about 1 mM to about 20 mM; dNTPs from about 0.001 mM to about 1 mM (including dATP, dCTP, dGTP, dTTp, dzGTP); ROX dye; a DNA polymerase (such as, but not limited to a thermostable DNA polymerase); NaN₃; VIP to facilitate hot start of DNA polymerase; KCl from about 20 mM to about 100 mM, to improve the specificity of the reaction; Nitro Red from about 0.01 mM to about 0.10 mM to monitor the addition of master mix when setting up reactions.

The present disclosure also provides kits for determining the copy number of a gene and/or for performing a CoP'ed method. A kit of the disclosure can comprise: at least a pair of target gene specific primers, a background sequence, at least a pair of primers specific to the background sequence, optionally, a pair of primers specific to a control or reference sequence, a DNA polymerase, dNTP's, MgCl₂ and one or more buffers.

A kit of the disclosure can also comprise one or more probes, wherein the probe comprises a nucleic acid sequence operable to selectively hybridize to: a target nucleic acid sequence, a reference/control nucleic acid sequence and/or to an amplicon or a fragment of an amplicon, including a target gene amplicon or a fragment thereof, a reference/control amplicon or fragment, and in some embodiments, and optionally to hybridize selectively to a background sequence, an amplicon or a fragment of an amplicon of a background sequence. Probes in a kit of the disclosure can include probes to perform a 5′ nuclease assay and/or one or more probes to detect the products of amplification. In some embodiments, one or more probe of a kit of the disclosure is labeled. In some embodiments, one or more probes of a kit of the disclosure is a dual labeled probe. In some embodiments, one or more of the probes of a kit of the disclosure is labeled with a fluor and a quencher. In some embodiments, each probe of a kit is dually labeled with a different fluor and a different quencher. In some embodiments, each probe of a kit is dually labeled with a different fluor and the same quencher.

A kit of the disclosure can also comprise one or more reagents for preparing crude cell lysates and/or reagents for extracting, isolating and/or purification of nucleic acids from a sample. Additional components can comprise particles with affinity for nucleic acids and/or solid supports with affinity for nucleic acids, one or more wash buffers, binding enhancers, binding solutions, polar solvents, alcohols, elution buffers, filter membranes and/or columns for isolation of DNA/RNA.

A kit for CoP'ed PCR can also comprise one or more of the following including: glycerol from about 3% to about 25%; gelatin from about 0.01% up to about 2%, such as bovine gelatin, fish gelatin; Tween 20 from about 0.001% to about 0.1%; Tris-HCL from about 0.01M to about 0.1M; MgCl₂ from about 1 mM to about 20 mM; Brij35; dNTPs from about 0.001 mM to about 1 mM (including dATP, dCTP, dGTP, dTTP, dzGTP); ROX dye; a DNA polymerase such as a thermostable DNA polymerase; a Taq polymerase; NaN₃; an antibody; VIP; KCl from about 20 mM to about 100 mM; Nitro Red from about 0.01 mM to about 0.10 mM.

A kit may further comprise reagents for downstream processing of an isolated nucleic acid and may include without limitation at least one RNase inhibitor; at least one cDNA construction reagents (such as reverse transcriptase); one or more reagents for amplification of RNA, one or more reagents for amplification of DNA including primers, reagents for purification of DNA, probes for detection of specific nucleic acids.

One or more compositions of a kit can be lyophilized. In some embodiments, all compositions of a kit of the disclosure will be lyophilized. In some embodiments, a kit of the disclosure with one or more lyophilized agents will be supplied with a re-constitution buffer

Reagents and components of kits may be comprised in one or more suitable container means. A container means may generally comprise at least one vial, test tube, flask, bottle, syringe or other container means, into which a component may be placed, and preferably, suitably aliquoted. Where there are more than one component in a kit they may be packaged together if suitable or the kit will generally contain a second, third or other additional container into which the additional components may be separately placed. However, in some embodiments, certain combinations of components may be packaged together comprised in one container means. A kit can also include a means for containing any reagent containers in close confinement for commercial sale. Such containers may include injection or blow-molded plastic containers into which the desired vials are retained.

A kit of the disclosure may also include instructions for employing the kit components and may also have instructions for the use of any other reagent not included in the kit. Instructions can include variations that can be implemented.

Some terms that are used in the specification are described below.

The term “isolation” refers to the act or process of removing, extracting or isolating a substance from a mixture. For e.g., isolating a biomolecule such as a nucleic acid, DNA, RNA, protein from a cell; a cell from a tissue/organism, a tissue from an organ/part, a tissue/cell/lysate from a tumor/cancer/diseases tissue, a lysate from a cell/tissue; and/or isolating one component from an environment of several components such as cellular components, and/or other materials in a sample. An “isolated” substance/nucleic acid may have significantly decreased quantities of other components that it was present in, and in some embodiments may be substantially pure or may be entirely pure (devoid of any contaminants, i.e., “purified”).

“Isolated” or “purified” generally refers to isolation of a substance (compound, polynucleotide, protein, polypeptide, polypeptide composition) such that the substance comprises a significant percent (e.g., greater than 2%, greater than 5%, greater than 10%, greater than 20%, greater than 50%, or more, sometimes more than 90%, 95% or 99%) of the sample in which it resides. In certain embodiments, a substantially purified component comprises at least 50%, 80%-85%, or 90-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in a higher proportion than it is naturally found.

The term “cells” refers to the smallest structural unit of an organism that is capable of independent functioning, consisting of one or more nuclei, cytoplasm, and various organelles, all surrounded by a semipermeable cell membrane.

The terms “ambient conditions” and “room temperature” are interchangeable and refer to common, prevailing, and uncontrolled atmospheric and weather conditions in a room or place.

“Hybridization” refers to a process in which single-stranded nucleic acids with complementary or near-complementary base sequences interact to form hydrogen-bonded complexes called hybrids. Hybridization reactions are sensitive and selective. “Selective hybridization” refers to the ability of single stranded nucleic acid molecules (such as primers, primer pairs and/or probes described herein) to selectively and specifically hybridize to complementary sequences in a target gene (or background gene, or reference/control gene) that the primer or probe is designed for and not to any other gene sequence. In vitro, the specificity of hybridization (i.e., stringency) is controlled by factors such as the concentrations of salt or formamide in pre-hybridization and hybridization solutions and by the hybridization temperature. In some embodiments, stringency may be increased by reducing the concentration of salt, increasing the concentration of formamide, and/or by raising the hybridization temperature. For example, high stringency conditions could occur at about 50% formamide at 37° C. to 42° C. Reduced stringency conditions could occur at about 35% to 25% formamide at 30° C. to 35° C. Some examples of stringency conditions for hybridization are also described in Sambrook, J., 1989, Molecular Cloning A Laboratory Manual, Cold Spring Harbor Laboratory Press, Cold Spring Harbor, N.Y. Generally, the temperature for hybridization is about 5-10° C. less than the melting temperature (Tm) of a hybrid nucleic acid.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, “amplification” or “amplify” and the like refers to a process that results in an increase in the copy number of a molecule or set of related molecules, to the production of multiple copies of a nucleic acid template, or the production of multiple nucleic acid sequence copies that are complementary to the nucleic acid template. Amplification can encompass a variety of chemical and enzymatic processes including without limitation, a polymerase chain reaction (PCR), a strand displacement amplification reaction, a transcription mediated amplification reaction, a nucleic acid sequence-based amplification reaction, a rolling circle amplification reaction, or a ligase chain reaction. According to certain embodiments, following at least one amplification cycle, the amplification products can be detected by sequence or by separation based on their molecular weight or length or mobility, for example.

The term “amplifying” that typically refers to an “exponential” increase in target nucleic acid may be used herein to describe both linear and exponential increases in the numbers of a select target sequence of nucleic acid. The term “amplification reaction mixture” and/or “master mix” may refer to an aqueous solution comprising the various (some or all) reagents used to amplify a target nucleic acid. Such reactions may also be performed using solid supports (e.g., an array). The reactions may also be performed in singleplex, duplex or multiplex format as desired by the user. These reactions typically include enzymes, aqueous buffers, salts, amplification primers, target nucleic acid, and nucleoside triphosphates. Depending upon the context, the mixture can be either a complete or incomplete amplification reaction mixture. The method used to amplify the target nucleic acid may be any available to one of skill in the art. Any in vitro means for multiplying the copies of a target sequence of nucleic acid may be utilized. These include linear, logarithmic, and/or any other amplification method. While this disclosure may generally discuss PCR as the nucleic acid amplification reaction, it is expected that other types of nucleic acid amplification reactions, including both polymerase-mediated amplification reactions (such as HDA, RPA, and RCA), as well as ligase-mediated amplification reactions (such as LDR, LCR, and gap-versions of each), and combinations of nucleic acid amplification reactions such as LDR and PCR (see for example U.S. Pat. No. 6,797,470) may also be suitable. Additional exemplary methods include polymerase chain reaction (PCR; see, e.g., U.S. Pat. Nos. 4,683,202; 4,683,195; 4,965,188; and/or 5,035,996), isothermal procedures (using one or more RNA polymerases (see, e.g., WO 2006/081222), strand displacement (see, e.g., U.S. Pat. No. RE39007E), partial destruction of primer molecules (see, e.g., WO2006087574)), ligase chain reaction (LCR) (see, e.g., Wu, et al., Genomics 4: 560-569 (1990)), and/or Barany, et al. PNAS USA 88:189-193 (1991)), Qβ RNA replicase systems (see, e.g., WO/1994/016108), RNA transcription-based systems (e.g., TAS, 3SR), rolling circle amplification (RCA) (see, e.g., U.S. Pat. No. 5,854,033; U.S. Pub. No. 2004/265897; Lizardi et al. Nat. Genet. 19: 225-232 (1998); and/or Banér et al. Nucleic Acid Res., 26: 5073-5078 (1998)), and strand displacement amplification (SDA) (Little, et al. Clin Chem 45:777-784 (1999)), among others. These systems, along with the many other systems available to the skilled artisan, may be suitable for use in amplifying target nucleic acids for use as described herein.

“Endpoint polymerase chain reaction” or “endpoint PCR” is a polymerase chain reaction method in which the presence or quantity of nucleic acid target sequence is detected after the PCR reaction is complete, and not while the reaction is ongoing. The term “end-point” measurement refers to a method where data collection occurs only once the reaction has been stopped, at the plateau phase of amplification.

The term “real-time” and “real-time continuous” are interchangeable and refer to a method where data collection occurs through periodic monitoring during the course of the polymerization reaction. Real-time methods combine amplification and detection into a single step.

“Real-time polymerase chain reaction” or “real-time PCR” is a polymerase chain reaction method in which the presence or quantity of nucleic acid target sequence is detected while the reaction is ongoing. In certain embodiments, the signal emitted by one or more probes present in a reaction composition is monitored during each cycle of the polymerase chain reaction as an indicator of synthesis of a primer extension product. In certain embodiments, fluorescence emitted during each cycle of the polymerase chain reaction is monitored as an indicator of synthesis of a primer extension product.

“Amplification efficiency” may refer to any product that may be quantified to determine copy number (e.g., the term may refer to a PCR amplicon, an LCR ligation product, and/or similar product). Reactions may be compared by carrying out at least two separate amplification reactions, each reaction being carried out in the absence and presence, respectively, of a reagent and/or step and quantifying amplification that occurs in each reaction.

Also provided are methods for amplifying a nucleic acid using at least one polymerase, at least one primer, dNTPs, and ligating and amplifying the target nucleic acid. In some embodiments of such methods, at least one primer is utilized. In certain embodiments, a nucleic acid amplification reaction mixture(s) comprising at least one polymerase, dNTPs, and at least one primer is provided. In other embodiments, methods for using such mixture(s) are provided. Target nucleic acids may be amplified using any of a variety of reactions and systems. Exemplary methods for amplifying nucleic acids include, for example, polymerase-mediated extension reactions. For instance, the polymerase-mediated extension reaction can be the polymerase chain reaction (PCR). In other embodiments, the nucleic acid amplification reaction is a multiplex reaction. For instance, exemplary methods for amplifying and detecting nucleic acids suitable for use as described herein are commercially available as TaqMan® (see, e.g., U.S. Pat. Nos. 4,889,818; 5,079,352; 5,210,015; 5,436,134; 5,487,972; 5,658,751; 5,210,015; 5,487,972; 5,538,848; 5,618,711; 5,677,152; 5,723,591; 5,773,258; 5,789,224; 5,801,155; 5,804,375; 5,876,930; 5,994,056; 6,030,787; 6,084,102; 6,127,155; 6,171,785; 6,214,979; 6,258,569; 6,814,934; 6,821,727; 7,141,377; and/or 7,445,900, all of which are hereby incorporated herein by reference in their entirety). TaqMan® assays are typically carried out by performing nucleic acid amplification on a target polynucleotide using a nucleic acid polymerase having 5′-3′ nuclease activity, a primer capable of hybridizing to said target polynucleotide, and an oligonucleotide probe capable of hybridizing to said target polynucleotide 3′ relative to said primer. In some embodiments, the oligonucleotide probe includes a detectable label (e.g., a fluorescent reporter molecule) and a quencher molecule capable of quenching the fluorescence of said reporter molecule. In certain embodiments, the detectable label and quencher molecule are part of a single probe. As amplification proceeds, the polymerase digests the probe to separate the detectable label from the quencher molecule. The detectable label (e.g., fluorescence) can be monitored during the reaction, where detection of the label corresponds to the occurrence of nucleic acid amplification (e.g., the higher the signal the greater the amount of amplification). Variations of TaqMan® assays (e.g., LNA™ spiked TaqMan® assay) are known in the art and would be suitable for use in the methods described herein.

Another exemplary system suitable for use as described herein utilizes double-stranded probes in displacement hybridization methods (see, e.g., Morrison et al. Anal. Biochem., 18:231-244 (1989); and/or Li, et al. Nucleic Acids Res., 30(2,e5) (2002)). In such methods, the probe typically includes two complementary oligonucleotides of different lengths where one includes a detectable label and the other includes a quencher molecule. When not bound to a target nucleic acid, the quencher suppresses the signal from the detectable label. The probe becomes detectable upon displacement hybridization with a target nucleic acid. Multiple probes may be used, each containing different detectable labels, such that multiple target nucleic acids may be queried in a single reaction.

Additional exemplary methods for amplifying and detecting target nucleic acids suitable for use as described herein involve “molecular beacons”, which are single-stranded hairpin shaped oligonucleotide probes. In the presence of the target sequence, the probe unfolds, binds and emits a signal (e.g., fluoresces). A molecular beacon typically includes at least four components: 1) the “loop”, an 18-30 nucleotide region which is complementary to the target sequence; 2) two 5-7 nucleotide “stems” found on either end of the loop and being complementary to one another; 3) at the 5′ end, a detectable label; and 4) at the 3′ end, a quencher dye that prevents the detectable label from emitting a single when the probe is in the closed loop shape (e.g., not bound to a target nucleic acid). Thus, in the presence of a complementary target, the “stem” portion of the beacon separates out resulting in the probe hybridizing to the target. Other types of molecular beacons are also known and may be suitable for use in the methods described herein. Molecular beacons may be used in a variety of assay systems. One such system is nucleic acid sequence-based amplification (NASBA®), a single step isothermal process for amplifying RNA to double stranded DNA without temperature cycling. A NASBA reaction typically requires avian myeloblastosis virus (AMV), reverse transcriptase (RT), T7 RNA polymerase, RNase H, and two oligonucleotide primers. After amplification, the amplified target nucleic acid may be detected using a molecular beacon. Other uses for molecular beacons are known in the art and would be suitable for use in the methods described herein.

The Scorpion system is another exemplary assay format that may be used in the methods described herein. Scorpion primers are bi-functional molecules in which a primer is covalently linked to the probe, along with a detectable label (e.g., a fluorophore) and a quencher. In the presence of a target nucleic acid, the detectable label and the quencher separate which leads to an increase in signal emitted from the detectable label. Typically, a primer used in the amplification reaction includes a probe element at the 5′ end along with a “PCR blocker” element (e.g., a hexethylene glycol (HEG) monomer (Whitcombe, et al. Nat. Biotech. 17: 804-807 (1999)) at the start of the hairpin loop. The probe typically includes a self-complementary stem sequence with a detectable label at one end and a quencher at the other. In the initial amplification cycles (e.g., PCR), the primer hybridizes to the target and extension occurs due to the action of polymerase. The Scorpion system may be used to examine and identify point mutations using multiple probes that may be differently tagged to distinguish between the probes. Using PCR as an example, after one extension cycle is complete, the newly synthesized target region will be attached to the same strand as the probe. Following the second cycle of denaturation and annealing, the probe and the target hybridize. The hairpin sequence then hybridizes to a part of the newly produced PCR product. This results in the separation of the detectable label from the quencher and causes emission of the signal. Other uses for molecular beacons are known in the art and would be suitable for use in the methods described herein.

The nucleic acid polymerases that may be employed in the disclosed nucleic acid amplification reactions may be any that function to carry out the desired reaction including, for example, a prokaryotic, fungal, viral, bacteriophage, plant, and/or eukaryotic nucleic acid polymerase

The term “polymerase” refers to an enzyme that is capable of adding at least one nucleotide onto the 3′ end of a primer, or to a primer extension product, that is annealed to a target nucleic acid sequence. In certain embodiments, the nucleotide is added to the 3′ end of the primer in a template-directed manner In certain embodiments, the polymerase is capable of sequentially adding two or more nucleotides onto the 3′ end of the primer. A “DNA polymerase” catalyzes the polymerization of deoxynucleotides.

The term “thermostable polymerase” refers to a polymerase that retains its ability to add at least one nucleotide onto the 3′ end of a primer, or to a primer extension product, that is annealed to a target nucleic acid sequence at a temperature higher than 37° C. The term “non-thermostable polymerase” refers to a polymerase that does not retain its ability to add at least one nucleotide onto the 3′ end of a primer, or to a primer extension product, that is annealed to a target nucleic acid sequence at a temperature higher than 37° C.

As used herein, the term “DNA polymerase” refers to an enzyme that synthesizes a DNA strand de novo using a nucleic acid strand as a template. DNA polymerase uses an existing DNA or RNA as the template for DNA synthesis and catalyzes the polymerization of deoxyribonucleotides alongside the template strand, which it reads. The newly synthesized DNA strand is complementary to the template strand. DNA polymerase can add free nucleotides only to the 3′-hydroxyl end of the newly forming strand. It synthesizes oligonucleotides via transfer of a nucleoside monophosphate from a deoxyribonucleoside triphosphate (dNTP) to the 3′-hydroxyl group of a growing oligonucleotide chain. This results in elongation of the new strand in a 5′ to 3′ direction. Since DNA polymerase can only add a nucleotide onto a pre-existing 3′-OH group, to begin a DNA synthesis reaction, the DNA polymerase needs a primer to which it can add the first nucleotide. Suitable primers may comprise oligonucleotides of RNA or DNA, or chimeras thereof (e.g., RNA/DNA chimerical primers). The DNA polymerases may be a naturally occurring DNA polymerases or a variant of natural enzyme having the above-mentioned activity. For example, it may include a DNA polymerase having a strand displacement activity, a DNA polymerase lacking 5′ to 3′ exonuclease activity, a DNA polymerase having a reverse transcriptase activity, or a DNA polymerase having an endonuclease activity.

Suitable nucleic acid polymerases may also comprise holoenzymes, functional portions of the holoenzymes, chimeric polymerase, or any modified polymerase that can effectuate the synthesis of a nucleic acid molecule. Within this disclosure, a DNA polymerase may also include a polymerase, terminal transferase, reverse transcriptase, telomerase, and/or polynucleotide phosphorylase. Non-limiting examples of polymerases may include, for example, T7 DNA polymerase, eukaryotic mitochondrial DNA Polymerase γ, prokaryotic DNA polymerase I, II, III, IV, and/or V; eukaryotic polymerase , , , , , η, , , and/or; E. coli DNA polymerase I; E. coli DNA polymerase III alpha and/or epsilon subunits; E. coli polymerase IV, E. coli polymerase V; T. aquaticus DNA polymerase I; B. stearothermophilus DNA polymerase I; Euryarchaeota polymerases; terminal deoxynucleotidyl transferase (TdT); S. cerevisiae polymerase 4; translesion synthesis polymerases; reverse transcriptase; and/or telomerase. Non-limiting examples of suitable thermostable DNA polymerases that may be used include Taq, Tfl, Pfu, and Vent™ DNA polymerases, any genetically engineered DNA polymerases, any having reduced or insignificant 3′ to 5′ exonuclease activity (e.g., SuperScript™ DNA polymerase), and/or genetically engineered DNA polymerases (e.g., those having the active site mutation F667Y or the equivalent of F667Y (e.g., in Tth), AmpliTaq®FS, ThermoSequenase™), Therminator I, Therminator II, Therminator III, Therminator Gamma (all available from NEB), and/or any derivatives and fragments thereof. Other nucleic acid polymerases may also be suitable as would be understood by one of skill in the art.

In another aspect, the present disclosure provides reaction mixtures for amplifying a nucleic acid sequence of interest (e.g., a target sequence, a background sequence and/or a control/reference sequence). In some embodiments, the reaction mixture may further comprise a signal-generating compound (SGC) and/or detectable label. The methods may also include one or more steps for detecting the SGC and/or detectable label to quantitate the amplified nucleic acid.

A SGC may be a substance that is itself detectable in an assay of choice, or capable of reacting to form a chemical or physical entity (e.g., a reaction product) that is detectable in an assay of choice. Representative examples of reaction products include precipitates, fluorescent signals, compounds having a color, and the like. Representative SGC include e.g., bioluminescent compounds (e.g., luciferase), fluorophores (e.g., below), bioluminescent and chemiluminescent compounds, radioisotopes (e.g., ¹³¹I, ¹²⁵I, ¹⁴C, ³H, ³⁵S, ³²P and the like), enzymes (e.g., below), binding proteins (e.g., biotin, avidin, streptavidin and the like), magnetic particles, chemically reactive compounds (e.g., colored stains), labeled oligonucleotides; molecular probes (e.g., CY3, Research Organics, Inc.), and the like. Representative fluorophores include fluorescein isothiocyanate, succinyl fluorescein, rhodamine B, lissamine, 9,10-diphenlyanthracene, perylene, rubrene, pyrene and fluorescent derivatives thereof such as isocyanate, isothiocyanate, acid chloride or sulfonyl chloride, umbelliferone, rare earth chelates of lanthanides such as Europium (Eu) and the like. Representative SGC's useful in a signal generating conjugate include the enzymes in: IUB Class 1, especially 1.1.1 and 1.6 (e.g., alcohol dehydrogenase, glycerol dehydrogenase, lactate dehydrogenase, malate dehydrogenase, glucose-6-phosphate dehydrogenase, glyceraldehyde-3-phosphate dehydrogenase and the like); IUB Class 1.11.1 (e.g., catalase, peroxidase, amino acid oxidase, galactose oxidase, glucose oxidase, ascorbate oxidase, diaphorase, urease and the like); IUB Class 2, especially 2.7 and 2.7.1 (e.g., hexokinase and the like); IUB Class 3, especially 3.2.1 and 3.1.3 (e.g., alpha amylase, cellulase, β-galacturonidase, amyloglucosidase, -glucuronidase, alkaline phosphatase, acid phosphatase and the like); IUB Class 4 (e.g., lyases); IUB Class 5 especially 5.3 and 5.4 (e.g., phosphoglucose isomerase, trios phosphatase isomerase, phosphoglucose mutase and the like.) SGCs may also generate products detectable by fluorescent and chemiluminescent wavelengths, e.g., sequencing dyes, luciferase, fluorescence emitting metals such as ¹⁵²Eu, or others of the lanthanide series; compounds such as luminol, isoluminol, acridinium salts, and the like; bioluminescent compounds such as luciferin; fluorescent proteins (e.g., GFP or variants thereof); and the like. Attaching certain SGC to agents can be accomplished through metal chelating groups such as EDTA. The subject SGC shares the common property of allowing detection and/or quantification of an attached molecule. SGCs are optionally detectable using a visual or optical method; preferably, with a method amenable to automation such as a spectrophotometric method, a fluorescence method, a chemiluminescent method, an electrical nanometric method involving e.g., a change in conductance, impedance, resistance and the like and a magnetic field method. Some SGCs are optionally detectable with the naked eye or with a signal detection apparatus. Some SGCs are not themselves detectable but become detectable when subject to further treatment. The SGC can be attached in any manner (e.g., through covalent or non-covalent bonds) to a binding agent of interest (e.g., an antibody or a PDZ polypeptide). SGCs suitable for attachment to agents such as antibodies include colloidal gold, fluorescent antibodies, Europium, latex particles, and enzymes. The agents that bind to NS1 and NP can each comprise distinct SGCs. For example, red latex particles can be conjugated to anti-NS1 antibodies and blue latex particles can be conjugated to anti-NP antibodies. Other detectable SGCs suitable for use in a lateral flow format include any moiety that is detectable by spectroscopic, photochemical, biochemical, immunochemical, electrical, optical, chemical, or other means. For example, suitable SGCs include biotin for staining with labeled streptavidin conjugate, fluorescent dyes (e.g., fluorescein, Texas red, rhodamine, green fluorescent protein, and the like), radiolabels, enzymes (e.g., horseradish peroxidase, alkaline phosphatase and others commonly used in an ELISA), and colorimetric SGCs such as colloidal gold or colored glass or plastic (e.g., polystyrene, polypropylene, latex beads). Patents that described the use of such labels include U.S. Pat. Nos. 3,817,837; 3,850,752; 3,939,350; 3,996,345; 4,277,437; 4,275,149; and 4,366,241. See also Handbook of Fluorescent Probes and Research Chemicals (6th Ed., Molecular Probes, Inc., Eugene Oreg.). Radiolabels can be detected using photographic film or scintillation counters, fluorescent markers can be detected using a photodetector to detect emitted light.

Similarly, the term “detectable label” may refer to any of a variety of signaling molecules indicative of amplification. For example, SYBR GREEN and other DNA-binding dyes are detectable labels. Such detectable labels may comprise or may be, for example, nucleic acid intercalating agents or non-intercalating agents. As used herein, an intercalating agent is an agent or moiety capable of non-covalent insertion between stacked base pairs of a double-stranded nucleic acid molecule. A non-intercalating agent is one that does not insert into the double-stranded nucleic acid molecule. The nucleic acid binding agent may produce a detectable signal directly or indirectly. The signal may be detectable directly using, for example, fluorescence and/or absorbance, or indirectly using, for example, any moiety or ligand that is detectably affected by proximity to double-stranded nucleic acid is suitable such as a substituted label moiety or binding ligand attached to the nucleic acid binding agent. It is typically necessary for the nucleic acid binding agent to produce a detectable signal when bound to a double-stranded nucleic acid that is distinguishable from the signal produced when that same agent is in solution or bound to a single-stranded nucleic acid. For example, intercalating agents such as ethidium bromide fluoresce more intensely when intercalated into double-stranded DNA than when bound to single-stranded DNA, RNA, or in solution (see, e.g., U.S. Pat. Nos. 5,994,056; 6,171,785; and/or 6,814,934). Similarly, actinomycin D fluoresces red fluorescence when bound to single-stranded nucleic acids, and green when bound to double-stranded nucleic acids. And in another example, the photoreactive psoralen 4-aminomethyle-4-5′8-trimethylpsoralen (AMT) has been reported to exhibit decreased absorption at long wavelengths and fluorescence upon intercalation into double-stranded DNA (Johnson et al. Photochem. & Photobiol., 33:785-791 (1981). For example, U.S. Pat. No. 4,257,774 describes the direct binding of fluorescent intercalators to DNA (e.g., ethidium salts, daunomycin, mepacrine and acridine orange, 4′6-diamidino-α-phenylindole). Non-intercalating agents (e.g., minor groove binders as described herein such as Hoechst 33258, distamycin, netropsin) may also be suitable for use. For example, Hoechst 33258 (Searle, et al. Nuc. Acids Res. 18(13):3753-3762 (1990)) exhibits altered fluorescence with an increasing amount of target. Minor groove binders are described in more detail elsewhere herein.

Other DNA binding dyes are available to one of skill in the art and may be used alone or in combination with other agents and/or components of an assay system. Exemplary DNA binding dyes may include, for example, acridines (e.g., acridine orange, acriflavine), actinomycin D (Jain, et al. J. Mol. Biol. 68:21 (1972)), anthramycin, BOBO™-1, BOBO™-3, BO-PRO™-1, cbromomycin, DAPI (Kapuseinski, et al. Nuc. Acids Res. 6(112): 3519 (1979)), daunomycin, distamycin (e.g., distamycin D), dyes described in U.S. Pat. No. 7,387,887, ellipticine, ethidium salts (e.g., ethidium bromide), fluorcoumanin, fluorescent intercalators as described in U.S. Pat. No. 4,257,774, GelStar® (Cambrex Bio Science Rockland Inc., Rockland, Me.), Hoechst 33258 (Searle and Embrey, 1990, Nuc. Acids Res. 18:3753-3762), Hoechst 33342, homidium, JO-PRO™-1, LIZ dyes, LO-PRO™-1, mepacrine, mithramycin, NED dyes, netropsin, 4′6-diamidino-α-phenylindole, proflavine, POPO™-1, POPO™-3, PO-PRO™-1, propidium iodide, ruthenium polypyridyls, S5, SYBR® Gold, SYBR® Green I (U.S. Pat. No. 5,436,134 and 5,658,751), SYBR® Green II, SYTOX blue, SYTOX green, SYTO® 43, SYTO® 44, SYTO® 45, SYTOX® Blue, TO-PRO®-1, SYTO® 11, SYTO® 13, SYTO® 15, SYTO® 16, SYTO® 20, SYTO® 23, thiazole orange (Aldrich Chemical Co., Milwaukee, Wis.), TOTO™-3, YO-PRO®-1, and YOYO®-3 (Molecular Probes, Inc., Eugene, Oreg.), among others. SYBR® Green I (see, e.g., U.S. Pat. Nos. 5,436,134; 5,658,751; and/or 6,569,927), for example, has been used to monitor a PCR reactions. Other DNA binding dyes may also be suitable as would be understood by one of skill in the art.

In some embodiments, SYBR green and other double stranded DNA binding dyes cannot be used for CoP'ed PCR methods described herein.

For use as described herein, one or more detectable labels and/or quenching agents may be attached to one or more primers and/or probes (e.g., detectable label). The detectable label may emit a signal when free or when bound to one of the target nucleic acids. The detectable label may also emit a signal when in proximity to another detectable label. Detectable labels may also be used with quencher molecules such that the signal is only detectable when not in sufficiently close proximity to the quencher molecule. For instance, in some embodiments, the assay system may cause the detectable label to be liberated from the quenching molecule. Any of several detectable labels may be used to label the primers and probes used in the methods described herein. As mentioned above, in some embodiments the detectable label may be attached to a probe, which may be incorporated into a primer, or may otherwise bind to the amplified target nucleic acid (e.g., a detectable nucleic acid binding agent such as an intercalating or non-intercalating dye). When using more than one detectable label, each should differ in their spectral properties such that the labels may be distinguished from each other, or such that together the detectable labels emit a signal that is not emitted by either detectable label alone. Exemplary detectable labels include, for instance, a fluorescent dye or fluorphore (e.g., a chemical group that can be excited by light to emit fluorescence or phosphorescence), “acceptor dyes” capable of quenching a fluorescent signal from a fluorescent donor dye, and the like. Suitable detectable labels may include, for example, fluorosceins (e.g., 5-carboxy-2,7-dichlorofluorescein; 5-Carboxyfluorescein (5-FAM); 5-HAT (Hydroxy Tryptamine); 5-Hydroxy Tryptamine (HAT); 6-JOE; 6-carboxyfluorescein (6-FAM); FITC; 6-carboxy-1,4-dichloro-2′, 7′-dichlorofluorescein (TET); 6-carboxy-1,4-dichloro-2′,4′, 5′, 7′-tetra-chlorofluorescein (HEX); 6-carboxy-4′,5′-dichloro-2′, 7′-dimethoxyfluorescein (JOE);); Alexa fluors (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethyl-rhodamine), Tetramethylrhodamine (TRITC), WT), Texas Red, Texas Red-X, VIC and other labels described in, e.g., US Pub. No. 2009/0197254 (incorporated herein by reference in its entirety), among others as would be known to those of skill in the art. Other detectable labels may also be used (see, e.g., US Pub. No. 2009/0197254 (incorporated herein by reference in its entirety)), as would be known to those of skill in the art. Any of these systems and detectable labels, as well as many others, may be used to detect amplified target nucleic acids.

As used herein, the term “nucleotide” or “nt” refers to a base-sugar-phosphate combination. Nucleotides are monomeric units of a nucleic acid molecule (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, 7-deaza-dGTP and 7-deaza-dATP. The term nucleotide as used herein also refers to dideoxyribonucleoside triphosphates (ddNTPs) and their derivatives. Examples of dideoxyribonucleoside triphosphates include, but are not limited to, ddATP, ddCTP, ddGTP, ddITP, and ddTTP.

As used herein, the phrase “nucleic acid molecule” refers to a sequence of contiguous nucleotides (riboNTPs, dNTPs or ddNTPs, or combinations thereof) of any length which can encode a full length polypeptide or a fragment of any length thereof, or which can be non-coding. As used herein, the terms “nucleic acid molecule” and “polynucleotide” can be used interchangeably and include both RNA and DNA.

As used herein, the term “oligonucleotide” refers to a synthetic or natural molecule comprising a covalently linked sequence of nucleotides which are joined by a phosphodiester bond between the 3′ position of the pentose of one nucleotide and the 5′ position of the pentose of the adjacent nucleotide.

As used herein, the term “nucleic acid” refers to polymers of nucleotides or derivatives thereof. As used herein, the term “target nucleic acid” refers to a nucleic acid that is desired to be amplified in a nucleic acid amplification reaction. For example, the target nucleic acid comprises a nucleic acid template. In some embodiments, a target nucleic acid may be the gene whose copy number is to be determined (e.g., a transgene of interest or a gene that has CNV).

As used herein, the term “sequence” refers to a nucleotide sequence of an oligonucleotide or a nucleic acid. Throughout the specification, whenever an oligonucleotide/nucleic acid is represented by a sequence of letters, the nucleotides are in 5′ to 3′ order from left to right. For example, if the polynucleotide contains bases Adenine, Guanine, Cytosine, Thymine, or Uracil, the polynucleotide sequence can be represented by a corresponding succession of letters A, G, C, T, or U), e.g., a DNA or RNA molecule. And, an oligonucleotide represented by a sequence (I)ₙ(A)ₙ wherein n=1, 2, 3, 4 and so on, represents an oligonucleotide where the 5′ terminal nucleotide(s) is inosine and the 3′ terminal nucleotide(s) is adenosine.

Sequence identity (also called homology) refer to similarity in sequence of two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (e.g., about 70% identity, preferably 75%, 80%, 85%, 90%, 95% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 90% identity at the amino acid level or at the nucleotide level. This definition also refers to the complement of a test sequence. Preferably, the identity exists over a region that is at least about 25, 50, or 100 residues in length, or across the entire length of at least one compared sequence. A preferred algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent conditions.

Oligonucleotides and/or polynucleotides can optionally be regarded as having “complementary” sequences if the same may hybridize to one another. The term “hybridization” typically refers to the process by which oligonucleotides and/or polynucleotides become hybridized to each other. The adjectival term “hybridized” refers to two polynucleotides which are bonded to each other by two or more sequentially adjacent base pairings. Typically, these terms refer to “specific hybridization”. Two oligonucleotides and/or polynucleotides may selectively (or specifically) hybridize to each other if they bind significantly or detectably to each other under stringent hybridization conditions when present in a complex polynucleotide mixture such as total cellular or library DNA. In some embodiments, for selective or specific hybridization, a positive signal is at least two times background, preferably 10 times background hybridization. Optionally, stringent conditions are selected to be about 5-10° C. lower than the thermal melting point for the specific sequence at a defined ionic strength pH. Stringent conditions are optionally in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary stringent hybridization conditions can be as following: 50% formamide, 5x SSC, and 1% SDS, incubating at 42° C., or, 5x SSC, 1% SDS, incubating at 65° C., with wash in 0.2x SSC, and 0.1% SDS at 65° C. Nucleic acids that do not hybridize to each other under stringent conditions are still substantially identical if the polypeptides which they encode are substantially identical. “Nonspecific hybridization” is used to refer to any unintended or insignificant hybridization, for example hybridization to an unintended polynucleotide sequence other than the intended target polynucleotide sequence. The unintended polynucleotide sequence can be on the same or different polynucleotide from the intended target. In some cases, the only intended hybridization can be from Watson-Crick base pairing between two polynucleotides. Other kinds of intended base pairings can include base pairing between corresponding analogs of such nucleotides or between iso-cytidine and iso-guanine. In some cases where hybridization is only intended between complementary bases, any bonding between non-complementary bases is considered to be non-specific hybridization.

In some embodiments, complementary sequences may be those that, when hybridized together, may be efficiently ligated to a third polynucleotide that has hybridized adjacently to it. Similarly, nucleotide residues can be regarded as complementary if when both are base-paired with each other within two hybridized polynucleotides, either nucleotide can be ligated in a template-driven ligation reaction when situated as the terminal nucleotide in its polynucleotide. Nucleotides that are efficiently incorporated by DNA polymerases opposite each other during DNA replication under physiological conditions are also considered complementary. In an embodiment, complementary nucleotides can form base pairs with each other, such as the A-T/U and G-C base pairs formed through specific Watson-Crick type hydrogen bonding between the nucleobases of nucleotides and/or polynucleotides positions antiparallel to each other. The complementarity of other artificial base pairs can be based on other types of hydrogen bonding and/or hydrophobicity of bases and/or shape complementarity between bases. In appropriate instances, polynucleotides can be regarded as complementary when the same may undergo cumulative base pairing at two or more individual corresponding positions in antiparallel orientation, as in a hybridized duplex. Optionally there can be “complete” or “total” complementarity between a first and second polynucleotide sequence where each nucleotide in the first polynucleotide sequence can undergo a stabilizing base pairing interaction with a nucleotide in the corresponding antiparallel position on the second polynucleotide. “Partial” complementarity describes polynucleotide sequences in which at least 20%, but less than 100%, of the residues of one polynucleotide are complementary to residues in the other polynucleotide. A “mismatch” is present at any position in the two opposed nucleotides that are not complementary. In some ligation assays, a polynucleotide can undergo substantial template-dependent ligation even when it has one or more mismatches to its hybridized template. Optionally, the polynucleotide has no more than 4, 3, or 2 mismatches, e.g., 0 or 1 mismatch, with its template. In some assays, the polynucleotide will not undergo substantial template-dependent ligation unless it is at least 60% complementary, e.g., at least about 70%, 80%, 85%, 90%, 95% or 100% complementary to its template.

“Degenerate”, with respect to a position in a polynucleotide that is one of a population of polynucleotides, means that the identity of the base of the nucleoside occupying that position varies among different members of the population. A population of polynucleotides in this context is optionally a mixture of polynucleotides within a single continuous phase (e.g., a fluid). The “position” can be designated by a numerical value assigned to one or more nucleotides in a polynucleotide, generally with respect to the 5′ or 3′ end. For example, the terminal nucleotide at the 3′ end of an extension probe may be assigned position 1. Thus in a pool of extension probes of structure 3′-XXXNXXXX-5′, the N is at position 4. A position is said to be k-fold degenerate if it can be occupied by nucleosides having any of k different identities. For example, a position that can be occupied by nucleosides comprising either of 2 different bases is 2-fold degenerate.

A “solid support”, as used herein, typically refers to a structure or matrix on or in which ligation and/or amplification reagents (e.g., nucleic acid molecules, microparticles, and/or the like) may be immobilized so that they are significantly or entirely prevented from diffusing freely or moving with respect to one another. The reagents can for example be placed in contact with the support, and optionally covalently or noncovalently attached or partially/completely embedded. The terms “microparticle,” “beads” “microbeads”, etc., refer to particles (optionally but not necessarily spherical in shape) having a smallest cross-sectional length (e.g., diameter) of 50 microns or less, preferably 10 microns or less, 3 microns or less, approximately 1 micron or less, approximately 0.5 microns or less, e.g., approximately 0.1, 0.2, 0.3, or 0.4 microns, or smaller (e.g., under 1 nanometer, about 1-10 nanometer, about 10-100 nanometers, or about 100-500 nanometers). Microparticles (e.g., Dynabeads from Dynal, Oslo, Norway) may be made of a variety of inorganic or organic materials including, but not limited to, glass (e.g., controlled pore glass), silica, zirconia, cross-linked polystyrene, polyacrylate, polymehtymethacrylate, titanium dioxide, latex, polystyrene, etc. Magnetization can facilitate collection and concentration of the microparticle-attached reagents (e.g., polynucleotides or ligases) after amplification, and facilitates additional steps (e.g., washes, reagent removal, etc.). In certain embodiments of the invention a population of microparticles having different shapes sizes and/or colors can be used. The microparticles can optionally be encoded, e.g., with quantum dots such that each microparticle can be individually or uniquely identified.

As used herein the term “reaction mixture” refers to the combination of reagents or reagent solutions, which are used to carry out a chemical analysis or a biological assay. In some embodiments, the reaction mixture comprises all necessary components to carry out a nucleic acid (DNA) synthesis/amplification reaction. As described above, such reaction mixtures may include at least one amplification primer pair suitable for amplifying a nucleic acid sequence of interest (e.g., target nucleic acid). As described above, such reaction mixtures may include at least one amplification primer pair suitable for amplifying a background nucleic acid sequence (e.g., background sequence). As described above, a suitable reaction mixture may also include a “master mix” containing the components (e.g., typically not including the primer pair) needed to perform an amplification reaction (e.g., detergent, magnesium, buffer components, etc.). Other embodiments of reaction mixtures are also contemplated herein as would be understood by one of skill in the art.

As used herein, the terms “reagent solution” or “solution suitable for performing a DNA synthesis reaction” refer to any or all solutions, which are typically used to perform an amplification reaction or DNA synthesis. They include, but are not limited to, solutions used in DNA amplification methods, solutions used in PCR amplification reactions, or the like. The solution suitable for DNA synthesis reaction may comprise buffer, salts, and/or nucleotides. It may further comprise primers and/or DNA templates to be amplified. One or more reagent solutions are typically included in the reactions mixtures or master mixes described herein.

As used herein, the term “primer” or “primer sequence” refers to a short linear oligonucleotide that hybridizes to a target nucleic acid sequence (e.g., a DNA template to be amplified) to prime a nucleic acid synthesis reaction. A primer polynucleotide or oligonucleotide has a free 3′-OH (or functional equivalent thereof) that can be extended by at least one nucleotide in a primer extension reaction catalyzed by a polymerase. In certain embodiments, primers may be of virtually any length, provided they are sufficiently long to hybridize to a target nucleic acid sequence of interest in the environment in which primer extension is to take place. In certain embodiments, primers are specific for a particular target nucleic acid sequence. In certain embodiments, primers are degenerate, e.g., specific for a set of target nucleic acid sequences. A primer may be a RNA oligonucleotide, a DNA oligonucleotide, or a chimeric sequence (e.g., comprising RNA and DNA). The primer may contain natural, synthetic, or modified nucleotides. Both the upper and lower limits of the length of the primer are empirically determined. The lower limit on primer length is the minimum length that is required to form a stable duplex upon hybridization with the target nucleic acid under nucleic acid amplification reaction conditions. Very short primers (usually less than 3 nucleotides long) do not form thermodynamically stable duplexes with target nucleic acid under such hybridization conditions. The upper limit is often determined by the possibility of having a duplex formation in a region other than the pre-determined nucleic acid sequence in the target nucleic acid. Generally, suitable primer lengths are in the range of about any of, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, (and so on) nucleotides in length.

The terms “primer set” or “set of primers” refer to two or more primers that are used as a set. In certain embodiments, a primer set may be designed to hybridize to sequences that flank a specific target nucleic acid sequence to be amplified. In certain embodiments, a primer set may be designed to hybridize to sequences that flank more than one different target nucleic acid sequence to be amplified.

Primers can be designed by the use of any of various software programs available and known in the art for developing amplification and/or multiplex systems. Exemplary programs include, PRIMER EXPRESS® software (Applied Biosystems, Foster City, Calif.) and Primer3 software (Rozen S et al. (2000), “Primer3 on the WWW for general users and for biologist programmers,” Krawetz S et al. (eds) Bioinformatics Methods and Protocols: Methods in Molecular Biology. Humana Press, Totowa, N.J., pp 365-386). In the example of the use of software programs, sequence information from a target or a background sequence can be imported into the software. The software then uses various algorithms to select primers that best meet the user's specifications for the targets.

In some embodiments, the terms “probe(s)”, “oligonucleotide(s)” and/or “primer(s)” may be interchangeable terms herein, so that any one of these may be taken as a reference to another. The terms “polynucleotide,” “oligonucleotide”, “probe”, “primer”, “template”, “nucleic acid” and the like may be taken to refer to a populations or pools of individual molecules that are substantially identical across their entire length or across a relevant portion of interest. For example, the term “template” may indicate a plurality of template molecules that are substantially identical, etc. In the case of polynucleotides that are degenerate at one or more positions, it will be appreciated that the degenerate polynucleotide may comprise a plurality of polynucleotide molecules, which have sequences that are substantially identical only at the nondegenerate position(s) and differ in sequence at the degenerate positions. Thus, reference to “a” polynucleotide (e.g., “a” primer, probe, oligonucleotide, template, etc.) may be taken to mean a population of substantially identical polynucleotide molecules, such that the plural nature of a population of substantially identical nucleic acid molecules need not be explicitly indicated, but may if so desired. These terms are also intended to provide adequate support for a claim that explicitly specifies a single polynucleotide molecule itself.

Some terms used in quantitation analysis of amplified PCR products are described here. “Amplicon” or “products of amplification” refers to a short segment of DNA generated by the PCR process. An “amplification plot” is a plot of fluorescence signal versus PCR cycle number. “Baseline” describes the initial cycles of PCR, in which there is little change in fluorescence signal. “Ct” fir “threshold cycle” describes the fractional cycle number at which the fluorescence passes the fixed threshold, “NTC” or “no template control” describes a sample that does not contain template. A “template” describes a target nucleic acid sequence or any nucleic acid sequence that is desired to be amplified. NTC is used. to verify amplification quality. “Nucleic acid target” also called “target template” is the DNA or RNA sequence that one desired to amplify. “Passive reference” describes a dye that provides an internal reference to which the reporter dye signal can be normalized during data analysis. Normalization is necessary to correct for forestallment fluctuations caused by changes in concentration or volume. A pass reference dye is typically included in all PCR reagent kits. “Rn” or “normalized reporter” describes the fluorescence emission intensity of the reporter dye divided by the fluorescence emission intensity of the passive reference dye. “Rn+” is the Rn value of a reaction containing all components, including the template. “Rn−” is the Rn value of an un-reacted sample. The Rn-value can be obtained from: either the early cycles of a real-time PCR run (those cycles prior to a detectable increase in fluorescence), OR from a reaction that does not contain any template. “ΔRn” (or delta Rn) is the magnitude of the signal generated by the given set of PCR conditions. The ΔRn value is determined by the following formula: (Rn+)−(Rn−). “Standard” is a sample of known concentration used to construct a standard curve. By running standards of varying concentrations, one can create a standard curve from which one can extrapolate the quantity of an unknown sample. “Threshold” is the average standard deviation of Rn for the early PCR cycles, multiplied by an adjustable factor, The threshold should he set in the region associated with an exponential growth of PCR product. “Unknown” refers to a sample containing an unknown quantity of template such as for example an unknown number of gene copy numbers. Typically quantitative PCR is performed on a sample to determine the quantity of a template.

As used herein, “real-time PCR” refers to the detection and quantitation of a DNA or a surrogate thereof in a sample. In some embodiments, the amplified segment or “amplicon” can be detected in real time using a 5′-nuclease assay, particularly the TaqMan® assay as described by e.g., Holland et al. (Proc. Natl. Acad. Sci. USA 88:7276-7280, 1991); and Heid et al. (Genome Research 6:986-994, 1996). For use herein, a TaqMan® nucleotide sequence to which a TaqMan® probe binds can be designed into the primer portion, or known to be present in DNA of a sample.

“Tₘ” refers to the melting temperature (temperature at which 50% of the oligonucleotide is a duplex) of an oligonucleotide determined experimentally or calculated using the nearest-neighbor thermodynamic values of SantaLucia J. et al. (Biochemistry 35:3555-62, 1996) for DNA. In general, the Tₘ of the TaqMan® probe is about 10 degrees above the Tₘ of amplification primer pairs. The Tₘ of the MGB probes is calculated using the SantaLucia method with factors correcting for the increased Tₘ due to MGB.

When a TaqMan® probe is hybridized to DNA or a surrogate thereof, the 5′-exonuclease activity of a thermostable DNA-dependent DNA polymerase such as SUPERTAQ® (a Taq polymerase from Thermus aquaticus, Ambion, Austin, Tex.) digests the hybridized TaqMan® probe during the elongation cycle, separating the fluor from the quencher. The reporter fluor dye is then free from the quenching effect of the quencher moiety resulting in a decrease in FRET and an increase in emission of fluorescence from the fluorescent reporter dye. One molecule of reporter dye is generated for each new molecule synthesized, and detection of the free reporter dye provides the basis for quantitative interpretation of the data. In real-time PCR, the amount of fluorescent signal is monitored with each cycle of PCR. Once the signal reaches a detectable level, it has reached the “threshold or cycle threshold (Ct).” A fluorogenic PCR signal of a sample can be considered to be above background if its Ct value is at least 1 cycle less than that of a no-template control sample. The term “Ct” represents the PCR cycle number when the signal is first recorded as statistically significant. Thus, the lower the Ct value, the greater the concentration of nucleic acid target. In the TaqMan® assay, typically each cycle almost doubles the amount of PCR product and therefore, the fluorescent signal should double if there is no inhibition of the reaction and the reaction was nearly 100% efficient with purified nucleic acid. Certain systems such as the ABI 7500, 7500FAST, 7700 and 7900HT Sequence Detection Systems (Applied Biosystems, Foster City, Calif.) conduct monitoring during each thermal cycle at a pre-determined or user-defined point.

Detection method embodiments using a TaqMan® probe sequence comprise combining the test sample with PCR reagents, including a primer set having a forward primer and a reverse primer, a DNA polymerase, and a fluorescent detector oligonucleotide TaqMan® probe, as well as dNTP's and a salt, to form an amplification reaction mixture; subjecting the amplification reaction mixture to successive cycles of amplification to generate a fluorescent signal from the detector probe; and quantitating the nucleic acid presence based on the fluorescent signal cycle threshold of the amplification reaction.

Protocols and reagents for means of carrying out other 5′-nuclease assays are well known to one of skill in the art, and are described in various sources. For example, 5′-nuclease reactions and probes are described in U.S. Pat. Nos. 6,214,979 issued Apr. 10, 2001; 5,804,375 issued Sep. 8, 1998; 5,487,972 issued Jan. 30, 1996; and 5,210,015 issued May 11, 1993, all to Gelfand et al.

Unless otherwise apparent from the context, any feature can be claimed in combination with any other, or be claimed as not present in combination with another feature. A feature can be any piece of information that can characterize an invention or can limit the scope of a claim, for example any variation, step, feature, property, composition, method, step, degree, level, component, material, substance, element, mode, variable, aspect, measure, amount, option, embodiment, clause, descriptive term, claim element or limitation.

The singular forms “a”, “an” and “the” include plural referents unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified.

Where necessary, ranges have been supplied, and those ranges are inclusive of all sub-ranges there between. Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

In this disclosure, the use of the singular can include the plural unless specifically stated otherwise or unless, as will be understood by one of skill in the art in light of the present disclosure, the singular is the only functional embodiment. Thus, for example, “a” may mean more than one, and “one embodiment” may mean that the description applies to multiple embodiments. The phrase “and/or” denotes a shorthand way of indicating that the specific combination is contemplated in combination and, separately, in the alternative.

It will be appreciated that there is an implied “about” prior to the temperatures, concentrations, times, etc. discussed in the present teachings, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

Unless specifically noted in the above specification, embodiments in the above specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

Generally, features described herein are intended to be optional unless explicitly indicated to be necessary in the specification. Non-limiting examples of language indicating that a feature is regarded as optional in the specification include terms such as “variation,” “where,” “while,” “when,” “optionally,” “include,” “preferred,” “especial,” “recommended,” “advisable,” “particular,” “should,” “alternative,” “typical,” “representative,” “various,” “such as,” “the like,” “can,” “may,” “example,” “embodiment” or “aspect” “in some,” “example,” “exemplary”, “instance”, “if” or any combination and/or variation of such terms.

Any indication that a feature is optional is intended provide adequate support (e.g., under 35 U.S.C. 112 or Art. 83 and 84 of EPC) for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” “not including”, “not”, “cannot,” or any combination and/or variation of such language.

Similarly, referents such as “a,” “an,” “said,” or “the,” are intended to support both single and/or plural occurrences unless the context indicates otherwise. For example “a dog” is intended to include support for one dog, no more than one dog, at least one dog, a plurality of dogs, etc. Non-limiting examples of qualifying terms that indicate singularity include “a single”, “one,” “alone”, “only one,” “not more than one”, etc. Non-limiting examples of qualifying terms that indicate (potential or actual) plurality include “at least one,” “one or more,” “more than one,” “two or more,” “a multiplicity,” “a plurality,” “any combination of,” “any permutation of,” “any one or more of,” etc. Claims or descriptions that include “or” between one or more members of a group are considered satisfied if one, more than one, or all of the group members are present in, employed in, or otherwise relevant to a given product or process unless indicated to the contrary or otherwise evident from the context.

In the claims, any active verb (or its gerund) are intended to indicate the corresponding actual or attempted action, even if no actual action occurs. For example, the verb “hybridize” and gerund form “hybridizing” and the like refer to actual hybridization or to attempted hybridization by contacting nucleic acid sequences under conditions suitable for hybridization, even if no actual hybridization occurs. Similarly, “detecting” and “detection” when used in the claims refer to actual detection or to attempted detection, even if no target is actually detected.

Furthermore, it is to be understood that the inventions encompass all variations, combinations, and permutations of any one or more features described herein. Any one or more features may be explicitly excluded from the claims even if the specific exclusion is not set forth explicitly herein. It should also be understood that disclosure of a reagent for use in a method is intended to be synonymous with (and provide support for) that method involving the use of that reagent, according either to the specific methods disclosed herein, or other methods known in the art unless one of ordinary skill in the art would understand otherwise. In addition, where the specification and/or claims disclose a method, any one or more of the reagents disclosed herein may be used in the method, unless one of ordinary skill in the art would understand otherwise.

All publications and patents cited in this specification are herein incorporated by reference in their entirety into this application as if each individual publication or patent were specifically and individually indicated to be incorporated by reference. Genbank® records referenced by GID or accession number, particularly any polypeptide sequence, polynucleotide sequences or annotation thereof, are incorporated by reference herein. The citation of any publication is for its disclosure prior to the filing date and should not be construed as an admission that the present invention is not entitled to antedate such publication by virtue of prior invention.

Where ranges are given herein, the endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Those skilled in the art will recognize, or be able to ascertain using no more than routine experimentation, many equivalents to the specific embodiments of the invention described herein. The scope of the present invention is not intended to be limited to this Description.

Certain embodiments are further described in the following examples. These embodiments are provided as examples only and are not intended to limit the scope of the claims in any way.

### EXAMPLES

Aspects of the present teachings can be further understood in light of the following examples, which should not be construed as limiting the scope of the present teachings in any way.

### Example 1

### Assays to Test Chromosome 21 Copy Numbers

Chromosome 21 copy number determination assays were performed to determine if there were 1, 2 or 3 copies of this chromosome. Trisomy of chromosome 21 is known to be the cause of Down's syndrome. The method developed herein can be adapted into methods for detecting and diagnosing the presence or absence of trisomy in chromosome 21.

In this example, Coriell human lymphoblast cultured cells were used to isolate genomic DNA (gDNA) to determine the copy number of chromosome 21 in aneuploidy samples.

Materials and Methods: Human aneuploidy gDNA was ordered from Coriell (Coriell Institute for Medical Research, Catalog Numbers: NA01201, 01921, 02571, 01416, 06061, 17102, 17201, 04375, 11419) and used for the chromosome copy number detection. 5 ng of human aneuploidy gDNA was added as a target DNA template and running with a CoP'ed master mix/reaction mix of the composition comprising a chromosome 21 nucleic acid specific target primer pair, a background sequence (called the KAZ plasmid) (Life technologies , Cat# 4308323, TaqMan® Exogenous Internal Positive Control Reagents)), a pair of primers specific to the background sequence, and a pair of control primers, and one or more of glycerol, gelatin, Tween 20, Tris-HCL (pH 9), MgCl₂, dATP, dCTP, dGTP, dTTp, dzGTP, ROX dye, AmpliTaq® DNA polymerase, NaN₃, VIP, KCl, Nitro Red with fast cycle specified by each SDS instruments such as 7500 fast and Vii7.

One Chromosome 21 target sequence or locus (ATP50, 21q2.11), and one chromosome X target sequence or locus (SH3BGRL, Xq3.3) were used to evaluate copy number of the aneuploidy samples. CoP'ed PCR was run by adding 10 pg KAZ plasmid and 1 μM primers in the final PCR.

In some embodiments a master-mix/reaction mix for a CoP'ed PCR method comprises one or more agents such as: glycerol from about 3% to about 25%; gelatin from about 0.01% up to about 2%, such as for example, bovine gelatin, fish gelatin; Tween 20 from about 0.001% to about 0.1%; Tris-HCL from about 0.01M to about 0.1M; MgCl₂ from about 1 mM to about 20 mM; dNTPs from about 0.001 mM to about 1 mM (including dATP, dCTP, dGTP, dTTp, dzGTP); ROX dye; a DNA polymerase (such as, but not limited to a thermostable DNA polymerase); NaN₃; VIP; KCl from about 20 mM to about 100 mM; and/or Nitro Red from about 0.01 mM to about 0.10 mM.

### Example 2

### CoP'ed PCR Reduces PCR Saturation of Target Nucleic Acid

Two different assays were tested with fixed amount of purified gDNA as target template and both standard PCR and CoP'ed PCR were run to compare the copy number separation upon the end of each PCR method.

Materials and Methods: Human aneuploidy gDNA ordered from Coriell is used for the chromosome copy# detection by taking 5 ng gDNA and running with a master mix as described in Example 1 with fast cycle. One Chromosome 21 (ATP50, 21q2.11) target sequence or locus, and one chromosome X (SH3BGRL, Xq3.3) target sequence or locus were used to evaluate copy# of aneuploidy samples. CoP'ed PCR was run by adding 10 pg KAZ plasmid as background sequence and 1 μM primers in the final PCR reaction mix.

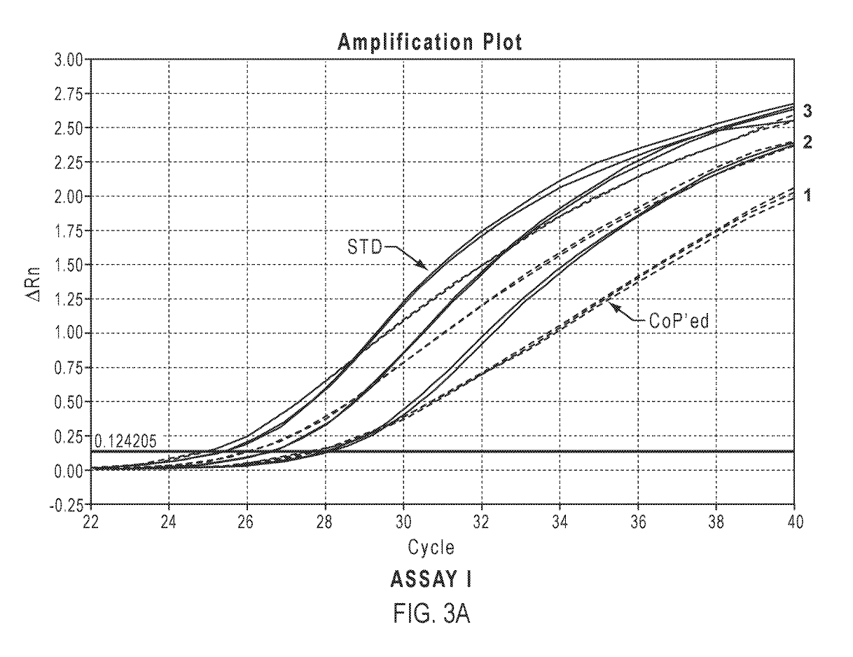

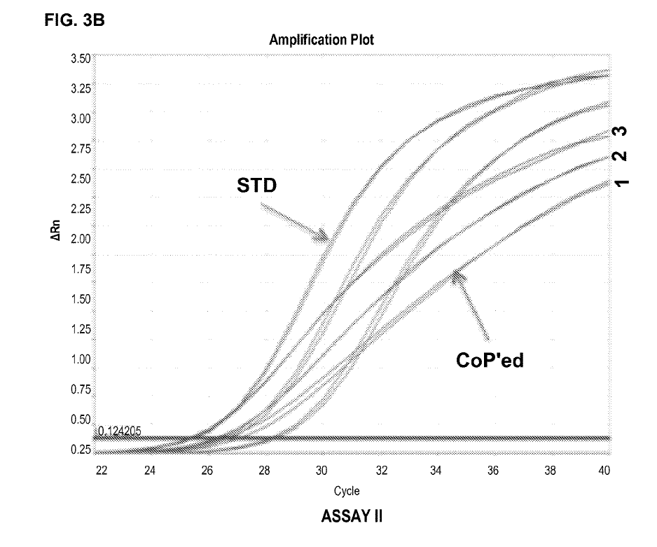

Results: FIGS. 3A and 3B depict the results of the CoP'ed PCR method as described above which reduced plateau of target PCR-gDNA. Two different assays were tested with a fixed amount of purified gDNA (10 ng/reaction). There is clear segregation of 1, 2 and 3 copies of target genes as seen from the amplification curves of the CoP'ed PCR which is absent in the standard PCR. At the end of PCR (Ct 40), the inventors were able to distinguish 1, 2 and 3 copies of chromosome 21 by the CoP'ed method comprising running the background PCR. In contrast to the CoP'ed PCR, in the standard PCR (referred to as STD PCR in FIGS.) the samples having 2 and 3 copies of the target gene (i.e., chromosome 21) are clustered together. No normalization was needed in this example since sample input (quantity of sample DNA) is the same in this test.

### Example 3

### CoP'ed PCR in the Presence of Crude Lysate Samples

To test if crude lysates affected the CoP'ed PCR method of the disclosure, target genes present on human gDNA were amplified by a CoP'ed method in the presence of crude lysates of samples (such as a corn leaf sample) to see if any agents of the crude lysate may affect the CoP'ed PCR method.

The next experiments that are contemplated by the inventors will be aimed at analyze DNA targets present in the crude lysate (rather than on human gDNA).

Materials and Methods: A crude corn lysate sample was obtained by using one punch (3 mm wide punch) of corn leaves and followed Sample-to-SNP protocol (Life Technologies, Cat# 4403081, also known as “Applied Biosystems® TaqMan® Sample-to-SNP™ Kit”). Briefly, for 3 mm leaf punch, add 50 μl lysis solution, then incubate at 95° C. for 3 minutes, cool down, then add 50 μl of DNA Stabilizing Solution. The target gene in this case was present in a human gDNA obtained from human cell lines. 2 μl of the crude corn leaf lysate was added to 10 ng human gDNA and a CoP'ed PCR was performed to test any carryover effect of crude lysate. For the background PCR a KAZ plasmid was used as a background sequence (as described in the previous example), at 10 pg per PCR and 1 μM background PCR primers in the final PCR reaction were used. The target genes used were 2 chromosome 21 target nucleic acid sequences described as the ATP50 and the TIAM1.

In this example method, varying amounts of input human gDNA sample ranging from 0.3 ng to 100 ng were tested (see corresponding copy numbers in the Results below). The PCR conditions followed default fast cycle, which is 95° C., 20 seconds followed by 40 cycles of 95° C., 1 second and 60° C., 20 second in each cycle. In the PCR mix, endogenous assay RNaseP (VIC-Tamra) was also included.

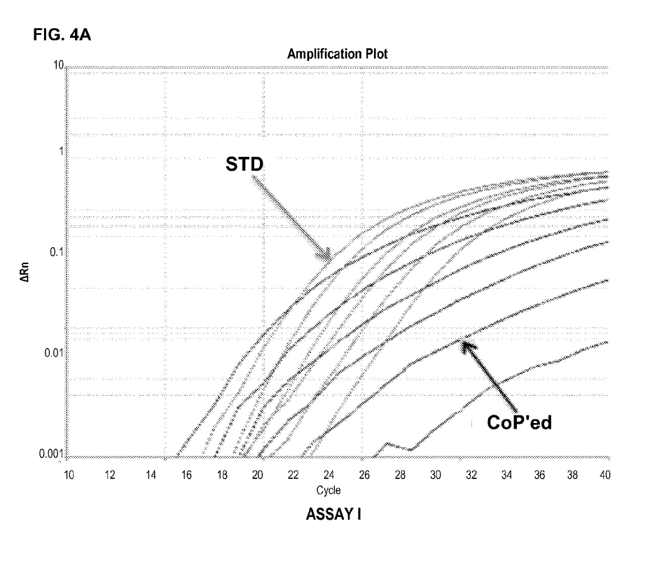

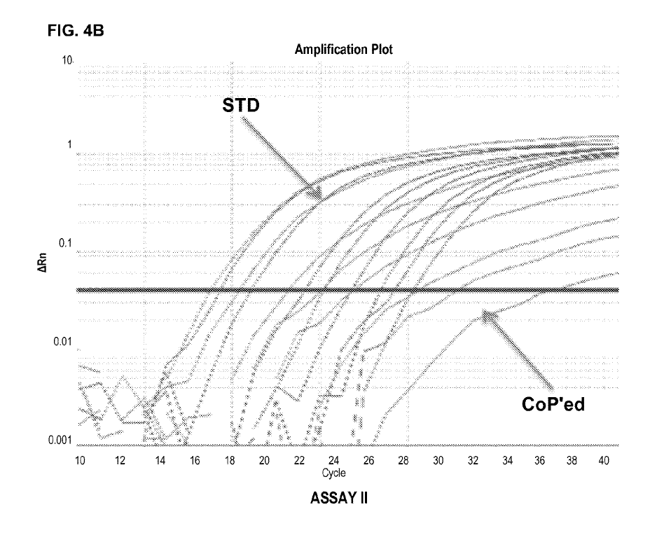

Results: FIGS. 4A and 4B depict results of the CoP'ed PCR assay using as a sample varying human gDNA and a corn crude lysate sample spiked-in to study effects, if any, on the CoP'ed PCR using KAZ as a background sequence. Different amount of gDNA (50 , 100, 200, 400 ,800, 1600, 3200 and 6400 copies of genome) were tested together with corn leaf crude lysate. A significantly superior and better separation of different copies of genome at the end of CoP'ed PCR as compared to with standard PCR (depicted as STD in the figures) is seen. The STD PCR readings show all data readings, regardless of the copy numbers, clustered together at the end. However, in the CoP'ed PCR in both examples of FIG. 4A and 4B clear separation of copy numbers is seen. In FIGS. 4A and 4B, the green colored curves depict the standard (STD) PCR and the black and pink curves depict the CoP'ed PCR. With CoP'ed PCR, the present inventors were able to achieve end point zygosity determination even when spiked with crude plant sample extracts.

Furthermore, as shown from the amplification curves in FIGS. 4A and 4B, CoP'ed PCR increased the separation of copy numbers using both end-point and also real-time qPCR analysis by control of the plateau of the amplified DNA.

CoP'ed PCR was unaffected by the addition of crude sample comprising the spiked corn leaf crude lysate into the gDNA template samples. A much superior separation of different copy numbers of target gene were seen at the end of the CoP'ed PCR as compared to the standard PCR (STD) in which all the various copy numbers clustered together at the end.

### Example 4

### CoP'ed PCR in Crude Samples Upon Data Normalization

Materials and Methods: The materials and methods were similar to those in Example 3. When amplification curves comprise comparison of fixed amount of gDNA as the input sample (as in Example 2), there is no need for sample input normalization.

However, since varying amounts of gDNA are used in this example as input template, sample input normalization is needed. Data is normalized by taking the ratio of Rn(target assay)/Rn(reference assay) to show the separation of copy# based on this ratio. This step is important for analysis of crude samples in applications such as zygosity/transgene determination in plants and crude/lysates of human/animal sample analysis, which will have varying amounts of target nucleic acid.

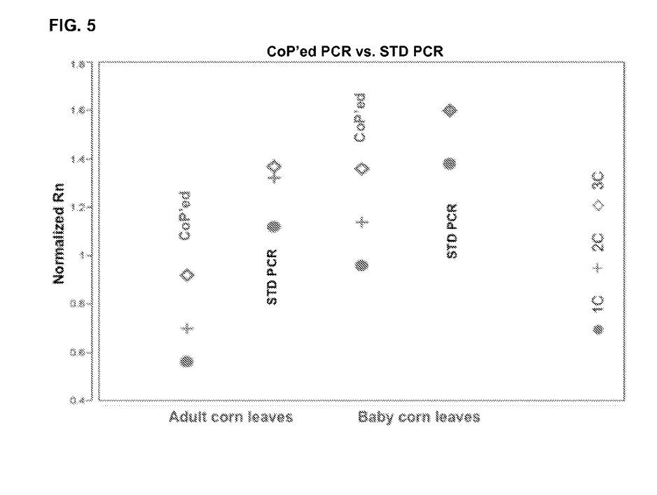

Results: FIG. 5 depicts an example CoP'ed PCR method for better end-point gene copy number separation with corn crude samples, CoP'ed PCR showed much better end-point zygosity in crude samples: with different amounts of gDNA spike-in to the corn crude samples, after normalization with calibration assay, significantly better separation was achieved by running background PCR compared with control.

After the normalization, CoP'ed PCR is able to distinguish the presence of 1, 2 and 3 copies from crude lysates of both adult and baby corn leaves as compared to STD PCR reactions for the same samples, in which there is no segregation of 2 and 3 copies.

### Example 5

### CoP'ed PCT improves Copy Number Separation for Both End-Point and Real-Time qPCR

Materials and Methods: 10 EDTA blood samples from 7 male and 3 female normal individuals were used to prepare crude blood lysate following Sample-to-SNP sample preparation protocol (as described previously). Briefly, 2 μl of blood was used in each lysate preparation and 2 μl of the lysis product (40 μl total) was used for each PCR. 5 pg of KAZ plasmid DNA and 0.5 μM of KAZ specific primers were used as background sequence and background primer pair. A master mix as described in Example 1 was used and default fast cycling condition was used for PCR. The target genes were 2 X-linked assays SHROOM4 and SH3BGRL and the endogenous control was a RnaseP assay used for sample gDNA input reference.

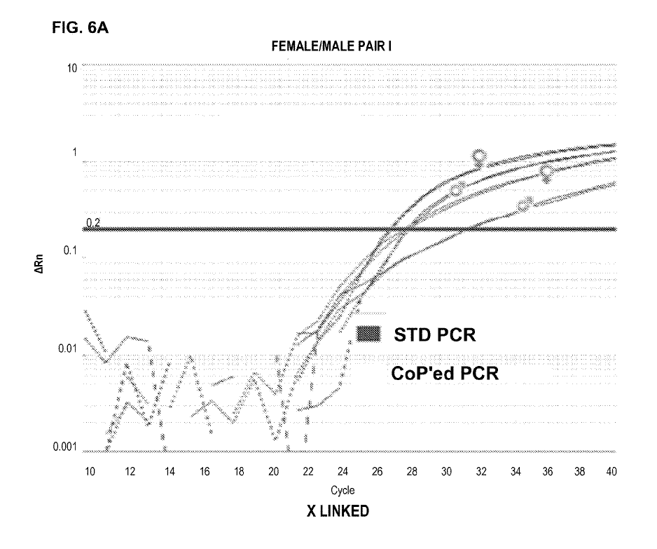

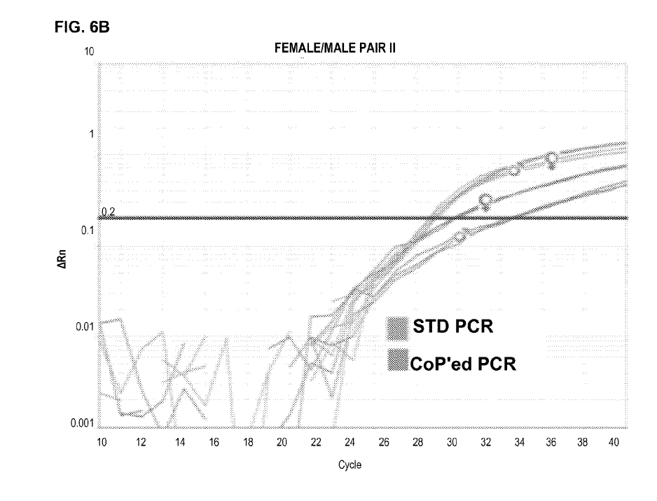

Results: FIGS. 6A and 6B depict an example CoP'ed PCR method showing improved copy number separation, for both real-time qPCR (Ct) and end-point Zygosity (Rn) methods by directly using human blood crude lysate in 2 separate X chromosome linked assays, SHROOM4 and SH3BGRL. The background sequence amplification of CoP'ed PCR competed with target PCR amplification for reagents and slowed down the amplification of the target sequence, therefore, enhancing the Ct as well as Rn difference among different copies. As shown in FIGS. 6A and 6B, Ct difference from 1 copy to 2 copy (male to female) can be as much as 4 Ct compared to standard PCR which is less than 1. The Rn based copy# separation also showed similar results, where the difference of detection of copy numbers was as much as 4 fold as well.

In PCR methods to evaluate the X chromosome copy#, without an input normalization, the inclusion of background PCR in a CoP'ed method, provided a clear separation of X chromosome copies between female and male using both end-point and real time qPCR analysis. With this improved separation, improved sensitivity of copy# detection can be achieved.

The present CoP'ed methods are especially useful for clinical samples with high heterogeneity containing small percentage of cancer or disease cells, since abnormal copy# detection is feasible even in such samples due to the copy # detection efficiency and sensitivity. Another application is for the Non-Invasive Prenatal Testing (NIPT). Due to the low percentage of fetal DNA (<10%) in maternal blood, regular qPCR methods are unable to detect fetal chromosome changes for Trisomies 21, 18 and 13 as well as monosomy X. In contrast, CoP'ed PCR methods of the present disclosure can amplify the difference and make the detection feasible.

Each embodiment disclosed herein may be used or otherwise combined with any of the other embodiments disclosed. Any element of any embodiment may be used in any embodiment. Although the invention has been described with reference to specific embodiments, it will be understood by those skilled in the art that various changes may be made and equivalents may be substituted for elements thereof without departing from the true spirit and scope of the invention. In addition, modification may be made without departing from the essential teachings of the invention.

The following references are incorporated by reference in their entirety:
1. Pierce, K. E. ,Sanchez, J. A. & Wangh, L. J. (2005) Proc.Natl.Acad.Sci. USA102, 8609-8614
2. Guthrie, P. A. I, Gaunt, T. R. & Day, L. N. M (2011) Nucleic Acids Research 1-12
3. Ingham, D. J., Beer, S. & Hansen, G (2001) BioTechniques 31: 132-140

What is claimed is:

1. A method of determining the number of copies of a gene comprising:
contacting a sample from which the number of copies of a target gene are to be determined with:
at least a first primer pair having the ability to selectively hybridize to portions of the target gene for which the number of copies are to be determined;
at least a second primer pair having the ability to selectively hybridize to portions of a background nucleic acid sequence; and
optionally, a third primer pair having the ability to selectively hybridize to portions of a reference nucleic acid sequence;
providing conditions for a polymerase chain reaction (PCR) to generate amplicons of:
a) the target gene or fragment thereof,
b) the background nucleic acid or fragment thereof, and
optionally, c) the reference nucleic acid or fragment thereof;
analyzing the amplified amplicons; and
determining the copy number of the target gene.

2. The method of claim 1, wherein determining the copy number comprises determining the Rn/ΔRn values.

3. The method of claim 1, wherein determining the copy number comprises end-point PCR.

4. The method of claim 1, wherein determining the copy number comprises determining the Ct values.

5. The method of claim 1, wherein determining the copy number comprises real-time qPCR.

6. The method of claim 1, wherein analyzing the amplicons comprises determining the Rn/ΔRn values or determining the Ct values.

7. The method of claim 1, further comprising using one or more probes, wherein the probes are labeled.

8. The method of claim 7, wherein the one or more probes are used for detecting amplification, quantifying amplification and for combinations thereof.

9. The method of claim 7, wherein the one or more probes are dually labeled.

10. The method of claim 9, wherein the one or more probes are labeled with a fluor and a quencher.

11. The method of claim 9, wherein each probe used is labeled with a different fluor and a different quencher, or with the same quencher and a different fluor.

12. The method of claim 7, wherein the probes and the primers are all contacted simultaneously with the sample for which gene copy number is to be determined.

13. The method of claim 12, wherein the PCR assay is a 5′nuclease assay.

14. The method of claim 7, wherein the one or more probes are contacted with the PCR reaction after amplification is complete to selectively hybridize to amplicons and to detect various amplicons generated in the PCR.

15. The method of claim 1, wherein the sample comprises a nucleic acid, an isolated nucleic acid, a gDNA, DNA, RNA, mRNA, a chromosome, a cell, a cell lysate, a plant derived sample, including samples from plant leaves, stems, seeds, germ, plant tissue derived crude lysates, bacterial samples, fungal samples, viral samples, animal samples, human samples, samples derived from cells, tissues, bodily fluids such as blood, plasma, serum, whole blood, lymph, sweat, semen, bone marrow, urine, fecal matter, saliva, buccal swab, milk, tumors, cancers, diseased tissues, samples obtained by biopsy, veterinary samples, skin samples, hair samples, crude lysates of any of the above, whole cells, and nucleic acids of any of the above.

16. The method of claim 1, wherein the sample is a crude cell lysate or a sample with varying amount of nucleic acid and wherein the sample is contacted with the third pair of PCR primers that have the ability to selectively hybridize to nucleic acid sequences in a reference nucleic acid.

17. A composition for a reaction mix for performing CoP'ed PCR comprising:
at least a pair of target gene specific primers;
a background nucleic acid sequence;
at least a pair of primers specific to the background sequence;
optionally, a pair of primers specific to a reference nucleic acid sequence;
a DNA polymerase;
dNTP's;
MgCl₂; and
one or more buffers.

18. The composition of claim 17, further comprising one or more probes, wherein the probe comprises a nucleic acid sequence operable to selectively hybridize to one or more of: a target nucleic acid sequence, a reference nucleic acid sequence, a background sequence, an amplicon or a fragment of an amplicon, wherein the amplicons can be a target gene amplicon or a fragment thereof, a reference amplicon or fragment thereof, a background sequence amplicon or a fragment thereof.

19. The composition of claim 17, further comprising one or more agents selected from a Taq Polymerase, VIP, antibodies, glycerol, gelatin, albumin, ROX dye, NAN₃, a detergent, Tween 20, Brij35, an emulsifier, a salt and combinations thereof.

20. A kit comprising:
at least a pair of target gene specific primers;
a background nucleic acid sequence;
at least a pair of primers specific to the background sequence;
optionally, a pair of primers specific to a reference nucleic acid sequence,
a DNA polymerase;
dNTP's;
MgCl₂;
one or more buffers; and
optionally, one or more probes,
wherein one or more of the components are comprised in one or more containers and having instructions for using the kit.
